(19) United States
(12) Patent Application Publication
(10) Pub. No.: US 2014/0044797 A1
(43) Pub. Date: Feb. 13, 2014

(54) PROSTACYCLIN AND ANALOGS THEREOF ADMINISTERED DURING SURGERY FOR PREVENTION AND TREATMENT OF CAPILLARY LEAKAGE

(71) Applicants: Par Johansson, Dosjebro (SE); Sisse Rye Ostrowski, Hellerup (DK)

(72) Inventors: Par Johansson, Dosjebro (SE); Sisse Rye Ostrowski, Hellerup (DK)

(73) Assignee: Rigshospitalet, Kobenhavn 0 (DK)

(21) Appl. No.: 14/113,090

(22) PCT Filed: Mar. 30, 2012
(86) PCT No.: PCT/DK2012/050100

§ 371 Date: Oct. 21, 2013

Related U.S. Application Data
(60) Provisional application No. 61/476,814, filed on Apr. 19, 2011.

(30) Foreign Application Priority Data
Apr. 19, 2011 (DK) PA 2011 70191

(51) Int. Cl. A61K 31/5585; A61K 45/06; A61K 31/5578

(52) U.S. Cl. CPC A61K 31/5585; A61K 31/5578; A61K 45/06
USPC 424/530; 562/501; 549/465; 514/469; 514/573; 514/14.9; 514/171; 514/562; 514/15.2

(57) ABSTRACT

The present invention relates to the novel use of prostacyclin analogs for prevention and/or treatment of capillary leakage during surgery. The treatment of the present invention mediates discrete or minimal effects on haemostasis and vasodilation. Thus the present invention provides prostacyclin and analogs thereof for treatment which prevents capillary leakage while minimizing the risk of bleeding. The present invention further provides pharmaceutical compositions and kits of parts comprising prostacyclin or analogs thereof, and methods for treatment.

### FIELD OF INVENTION

The present invention relates to the novel use of prostacyclin analogs during after surgery for prevention and/or treatment of capillary leakage. The treatment of the present invention mediates discrete or minimal effects on haemostasis and vasodilation. Thus the present invention provides prostacyclin and analogs thereof for treatment which prevents capillary leakage while minimizing the risk of bleeding during surgery. The present invention further provides pharmaceutical compositions and kits of parts comprising prostacyclin or analogs thereof, and methods for treatment or prevention of capillary leakage.

### BACKGROUND OF INVENTION

Endothelial cells line the interior part of blood vessels in the entire circulatory system, from the heart to the smallest capillary. On the luminal surface of the vascular endothelium is the glycocalyx (sugar coat), which is a layer of membrane-bound macro-molecules normally having a functional thickness of more than 1 μm, (Reitsma et al 2007) (in some regions it has been shown that the glycocalyx can be thicker than the endothelial cells themselves). The endothelial glycocalyx is also called “the plasma layer” or “the endothelial surface layer” (ELS).

The endothelial glycocalyx plays an important role in the control of blood flow and can exclude blood cells and macromolecular solutes of the blood, ensuring that these do no not come in contact with the endothelial cell membrane. Thus, the glycocalyx is actively involved in regulation of the vascular tone, fluid and solute exchange between blood and tissue, leukocyte migration, haemostasis as well as regulation of responses of coagulation and inflammation.

The endothelial glycocalyx consists of the carbohydrate moieties of membrane glycolipids and glycoproteins. It is considered to be connected to the endothelium through several “backbone” molecules, mainly proteoglycans and also glycoproteins. These form a network in which soluble molecules, either plasma- or endothelium-derived, are incorporated. A dynamic equilibrium exists between this layer of soluble components and the flowing blood, continuously affecting composition and thickness of the glycocalyx. The amount of plasma fixed within the endothelial surface layer (and therefore not quantitatively participating in the normal blood circulation) is approximately 700-1000 ml in humans, thus representing one third of the total plasma volume. The large dimension of the endothelial glycocalyx reveals a big and very important compartment of the circulation. However, as mentioned above, the volume is in dynamic and affected by changes in the glycocalyx, which can suffer from enzymatic or shear-induced shedding. Ischemia/reperfusion, proteases, tumor necrosis factor, oxidized low-density lipoprotein and atrial natriuretic peptide are additionally found to be capable of mediating degrading the glycocalyx. Diminution of the endothelial glycocalyx leads to platelet aggregation, leukocyte adhesion and an increase in endothelial permeability causing capillary leakage and tissue edema. For instance it has been shown that enzymatic (partial) removal and subsequent loss of permeability barrier function of the glycocalyx in rat myocardial capillaries leads to myocardial edema (van den Berg B M (2003)). In addition, proteoglycans which are that are part of the glycocalyx such as heparan sulfates play pivotal roles in inflammation.

Surgery, and the concurrent tissue trauma, inflammation, fluid administration, ischemia, and free oxygen radicals etc., has the potential to impair the general state of the endothelium and the endothelial glycocalyx which can result in severe post-surgical complications that lead to need of intensive care and increase the mortality of the patients. Such complications include capillary leakage resulting in abnormal extravasation of blood components and accumulation of fluid in the extravascular space (hyperpermeability) and tissue edema, tissue dysfunction in part due to both impaired blood flow and ischemia which may lead to clinical conditions such as abdominal compartment syndrome, single/multi organ failure or systemic capillary leak syndrome.

Prostacyclin is a naturally occurring prostaglandin released by healthy endothelial cells, which has potent vasodilatory activity and inhibitory activity of platelet aggregation. These effects have been used in a clinical setting by administering prostacyclin and analogs thereof in order to cause direct vasodilation of pulmonary and systemic arterial vascular beds and/or inhibit platelet aggregation. Because of these effects it is recommended not to administer prostacyclin in situations where there is an increased risk of excessive bleeding or hypotension, such as for example peptic ulcer, trauma, and intercranial bleeding. For the same reasons, prostacyclin is normally not used during surgery.

### SUMMARY OF THE INVENTION

The inventors of the present invention have surprisingly found that prostacyclin and analogs thereof when administered intra- and/or postoperatively in low doses are able to prevent or treat capillary leakage and at the same time only has minimal effects and on the haemostatic system and/or vasodilation, for instance as assessed by thrombelastography and/or whole blood aggregometry. Thus, in the present invention, prostacyclin is used for prevention of capillary leakage in a manner that minimises the risk of excessive surgical bleeding.

Accordingly, the present invention relates to a compound which is prostacyclin, or an analog thereof, for prevention of capillary leakage by intraoperative and/or post-operative administration.

In one embodiment of the present invention, the prevention of capillary leakage is mediated by protection of the endothelium including the glycocalyx.

In one embodiment of the present invention, a prostacyclin or an analog thereof is used for treatment by intraoperative and/or post-operative administration wherein the surgery is a gastroenterological, thoracic, orthopaedic, urological, gynaecological, plastic, cosmetic or reconstructive procedure. Such types of surgery may not be related to ischemia, cardiovascular diseases, transplantation or insertion of stents and grafts or trauma.

In one embodiment of the present invention, a compound which is prostacyclin or an analog thereof is selected from the group of PGI2, PGX, prostacyclin (Epoprostenol) or variants thereof, beraprost sodium, epoprostenol sodium, iloprost, iloprost in combination with bosentan, iloprost in combination with sildenafil citrate, treprostinil, pegylated treprostinil, treprostinil diethanolamine, treprostinil sodium, or derivatives thereof, and preferably from epoprostenol or iloprost, or derivatives thereof.

In one embodiment of the present invention, prostacyclin or an analog thereof is administered in doses within the range of 0.5 ng/kg/min to 4.0 ng/kg/min.

In another embodiment of the present invention, prostacyclin or an analog thereof is administered by parental administration, and preferably by continuous intravenous administration.

In a further embodiment of the present invention, prostacyclin or an analog thereof is used in a treatment period having a length of 15 minutes to 72 hours, and preferably 60 to 120 min.

In yet another embodiment of the present invention, prostacyclin or an analog thereof is used in a treatment period having a length of 15 to 360 min, and preferably 60 to 120 min.

In another embodiment the treatment period wherein the compounds of the present invention are administered includes the intraoperative treatment period and a post-operative period of up to 72 hours.

In one embodiment of the present invention, prostacyclin or an analog thereof is used in a treatment method which decreases or reduces risk of increased levels in a bodily sample of one or more markers, preferably glycocalyx markers selected from syndecan-1, glypican-1 and hyaluronan.

In one embodiment of the present invention, prostacyclin or an analog thereof is used in a treatment method which decreases or reduces risk of increased levels in a bodily sample of one or more biomarkers selected from adrenaline, noradrenaline, ICAM-1, E-selectin, Soluble fms-like tyrosine kinase-1 (also called sFlt-1 or sVEGF R1), sVE-cadherin, angiopoietin 1 (Ang-1), angiopoietin 2 (Ang-2), soluble thrombomodulin (sTM), soluble endothelial protein C receptor (sEPCR), protein C (PC), activated protein C (APC), Antithrombin III (AT) (Antithrombin III), tissue factor pathway inhibitor (TFPI), von Willebrand factor (vWF), tissue-type plasminogen activator (tPA), Factor XIII, histon-complexed DNA fragments, high-mobility group protein B1 (HMGB1), d-dimer, IL-6, sCD40L and sC5B9.

In one embodiment of the present invention, said treatment decreases or reduces risk of one or more of the following factors:
a. need for fluid administration perioperatively and/or post-surgery, or the volume of fluid administered perioperatively and/or post-surgery,
b. weight gain 12 hours, 24 hours, 48 hours and 72 hours post-surgery compared to pre-surgery,
c. incidence of abdominal compartment syndrome post-surgery,
d. need for supportive therapy such as intensive care therapy such pressor support (drugs such as noradrenaline, dobutamine), ventilatory support, dialysis and treatment for septic complications,
e. occurrence of single/multiple organ failures selected from the group of organ failures in the central nervous system, lungs, heart, gastrointestinal system, kidneys, liver and haematological systems,
f. coagulopathy.

In one embodiment of the present invention, the thrombelastography values measured during or after treatment as defined herein in a citrated blood sample activated by kaolin are within the ranges a) R between 3.0 to 8.0 minutes, b) Angle between 55° and 78°, and c) MA between 51 mm to 69 mm.

In one embodiment of the present invention, the treatment with prostacyclin or an analog thereof leads to discrete or minimal effects on the hemostatic system such as a significant inhibition of platelet aggregation (for instance measured by electrical impedance aggregometry) or coagulation of blood (for instance measured by thrombelastography (TEG)).

In another embodiment of the present invention, the treatment has discrete or minimal vasodilation effects on the microcirculation.

In another embodiment of the present invention, treatment using prostacyclin or analogs thereof leads to aggregation units measured by multiplate electrical impedance aggregometry during or after treatment in the range of 40 to 200.

The present invention further provides a pharmaceutical composition for treatment or prevention of capillary leakage which comprises prostacyclin or an analog thereof.

In one embodiment of the present invention, said pharmaceutical composition as defined herein mediates a protection of the endothelium including the glycocalyx.

In another embodiment of the present invention, said pharmaceutical composition as defined herein mediates a protection of the endothelial glycocalyx.

In one embodiment of the present invention, a pharmaceutical composition may comprise prostacyclin or an analog thereof in a dose of 0.375 μg to 750 μg, or may comprise a dose of prostacyclin or an analog thereof suitable for a period of treatment which is 15 min to 360 min, i.e. for intraoperative administration.

In one embodiment of the present invention, a pharmaceutical compositions comprises one or more second active ingredients, such as for example at least one second active ingredient which is an agonist or antagonist of adrenergic receptors, or such as for example at least one second active ingredient is Antithrombin III (AT), hydrocortisone, glucocorticoids, N-acetylcysteine or albumin.

The present invention further provides kits of parts for treatment or prevention of capillary leakage comprising prostacyclin or an analog thereof as defined herein or a pharmaceutical composition as herein.

In one embodiment of the present invention, the kit of parts as defined herein is for use in a method wherein prostacyclin or an analog as defined herein thereof mediates a protection of the endothelium including the glycocalyx.

Methods for treatment or prevention of capillary leakage are also aspects of the present invention. Such methods comprise a step wherein prostacyclin or an analog thereof as defined herein, or a pharmaceutical composition as defined herein, is administered intraoperatively and/or post-operatively by separate, sequential or simultaneous administration to an individual.

In one embodiment of the present invention, said the administration of prostacyclin or an analog thereof mediates a protection of the endothelium including the glycocalyx ad particularly the endothelial glycocalyx itself.

In one embodiment of the present invention, a method for treatment further comprises a step wherein one or more second active ingredients is administered separately, sequentially or simultaneously to an individual.

### DESCRIPTION OF DRAWINGS

FIG. 1: Recording haemostatic activity using Thrombelastography (TEG): Four parameters are routinely reported: R (reaction time) denotes the latency from the time at which the blood is placed in the cup until the clot begins to form; the angle (Angle) represents the progressive increase in clot strength; the maximum amplitude (MA) reflects the maximal clot strength; and lysis (Ly30) reflects clot lysis. The figure shows baseline or normal TEG values with samples obtained after 60- and 120 min of flolan (prostacyclin analog) infusion in healthy humans.

FIG. 2: Multiplate whole blood aggregometry is a platelet function test, (Multiplate®, Dynabyte Medical, Munich, Germany). This test is based on multiple electrode platelet aggregometry (MEA), which measures platelet aggregation in whole blood (WB) after stimulation with selective platelet agonists such as trombinactivated peptide (TRAP), ADP, ASPI and COLlagen. The increase of impedance by the attachment of platelets onto the Multiplate sensors is transformed to arbitrary aggregation units (AU) and plotted against time. Multiplate thereby allows analyzing the effect of treatments as defined herein. The figure shows baseline Multiplate AU values at selected time-points with samples obtained after 60- and 120 min of flolan (prostacyclin analog) infusion in healthy humans.

FIG. 3: A: Levels of thrombomodulin over time during and following prostacyclin administration in healthy individuals. B: Levels of Protein C over time during and following prostacyclin administration in healthy individuals.

FIG. 4: A: B: Levels of PAI-1 over time during and following prostacyclin administration in healthy individuals. B: Levels of Antithrombin over time during and following prostacyclin administration in healthy individuals.

FIG. 5: A: B: Levels of Histone-complexed DNA over time during and following prostacyclin administration in healthy individuals. B: Levels of HMGB1 over time during and following prostacyclin administration in healthy individuals.

FIG. 6: A: B: Levels of Syndecan-1 over time during and following prostacyclin administration in healthy individuals. B: Levels of TFPI over time during and following prostacyclin administration in healthy individuals.

### DEFINITIONS

The terms “antiaggregatory” and “antithrombotic” is used interchangeably and refers to the effect of compound(s) that reduces the platelets ability to interact in the clot building process and hence form thrombi.

The terms “coagulopathy” (also called clotting disorder and bleeding disorder) is any defect in the body's mechanism for coagulation and clot building, causing a predisposition either for too slow (hypocoagulability) or too quick (hypercoagulability) to coagulate. In some cases, a coagulopathy can present with both increased bleeding tendency and increased risk of thrombosis.

The term “critically ill”, herein also acutely ill, is meant to include any condition rendering the patient in need for intensive care therapy. Intensive care therapy may include but is not limited to induction of homeostasis, ventilation (eg. Mechanical ventilation), haemodialysis, vasopressor support, fluid support, parenteral nutrition, administration of red blood cell concentrates, fresh frozen plasma, platelet concentrates, whole blood, systemic antibiotic and/or antiviral and/or antifungal and/or antiprotozoic therapy, granulocyte infusion, T cell infusion, stem cell infusion, anticoagulant therapy including but not limited to administration of activated protein C, analogs or fragments hereof and/or antithrombin and/or tissue factor pathway inhibitor (TFPI) (and/or heparins, including low molecular weight heparins, and/or thrombin inhibitors, administration of corticosteroids, tight glycemic control.

The term “glycocalyx” as mentioned herein refers to the carbohydrate-rich layer which is covering the endothelial cells in healthy individuals. The glycocalyx comprises proteoglycans which can be soluble or linked to the endothelial cell membrane.

The term “glycosaminoglycan” as mentioned herein refers to long unbranched polysaccharides consisting of a repeating disaccharide unit. The repeating unit consists of a hexose (six-carbon sugar) or a hexuronic acid, linked to a hexosamine (six-carbon sugar containing nitrogen) such as N-acetylglucosamine or N-acetylgalactosamine. GAGs have a high charge density and exhibit marked diversity due to variations in the disaccharides and sites of sulfation. In addition to heparin and heparan sulfate, GAGs may consist of chondroitin or dermatan sulfates, keratan sulfate, or hyaluronan. Synonyms of glycosaminoglycan is GAG and mucopolysaccharide.

The term “glypican” as mentioned herein refers to the GPC family of heparan sulphate proteoglycans that are anchored to the cell-surface via a covalent linkage to glycosylphosphatidylinositol (GPI). The amino acid sequences of the six vertebrate glypican family members vary from being 17% to 63% identical. Heparan sulphate glycosaminoglycan chains are attached to the C-terminal part of the protein, near the GPI anchor and the cell membrane.

The term “hyaluronan” (also called hyaluronic acid or hyaluronate) as mentioned herein is an anionic, nonsulfated glycosaminoglycan which is a polymer of disaccharides, themselves composed of D-glucuronic acid and D-N-acetylglucosamine, linked via alternating β-1,4 and β-1,3 glycosidic bonds. Hyaluronan can be 25000 disaccharide repeats in length and polymers of hyaluronan can have a variety of sizes, which are typically in the range from 5000 to 20000000 Da in vivo. While it is abundant in extracellular matrices, hyaluronan also contributes to tissue hydrodynamics, movement and proliferation of cells, and participates in a number of cell surface receptor interactions.

The term “hypocoagulability” used herein will reflect a slower initiation phase (increased R), and/or reduced thrombin burst (decreased Angle) and/or reduced clot strength (reduced MA) as evaluated by TEG as compared to the normal reference.

The term “hypercoagulability” used herein will reflect an increased coagulation activity in the initiation phase (decreased R), and/or increased thrombin burst (increased Angle) and/or increased clot strength (increased MA) as evaluated by TEG as compared to the normal reference.

The term “hypovolemia” (also hypovolaemia) is a state of decreased blood volume; more specifically, decrease in volume of blood plasma. It is thus the intravascular component of volume contraction (or loss of blood volume).

The term “multi organ failure” (or MOF) is altered organ function in an acutely ill patient requiring medical intervention to achieve homeostasis; MOF includes as used herein TAMOF. MOF is also known as Multiple organ dysfunction syndrome (MODS).

The term “prostacyclin” refers to the lipid molecule prostacyclin (PGI2) which is a member of the family eicosanoids. The definition as used herein also includes prostacyclin analogs, or prostacyclin receptors agonists which have affinity for prostacyclin receptors and may be able to mediate functions similar to the function of prostacyclin.

“Prostacyclin analog” refers to a drug that can initiate a physiological or a pharmacological response characteristic of prostacyclin. Prostacyclin analogs according to the present invention includes, but are not limited to, compounds that have affinity for the prostacyclin receptor and are capable of activating a prostacyclin receptor response in a manner similar to prostacyclin.

The term “protection” refers to reduction, ameliorating, alleviating or relieving degradation of the endothelium and the glyocalyx, the endothelium and/or glycocalyx themselves; delaying the progression of the endothelial cell damage, glycocalyx degradation; increasing the production of the glycocalyx and components of the glycocalyx, and/or reducing the risk of, or preventing the degradation of the glycocalyx. The term “shedding” of the glycocalyx is herein referred to as degradation of the glycocalyx.

The term “proteoglycan” refers to proteins that are heavily glycosylated. The basic proteoglycan unit consists of a protein with one or more covalently attached glycosaminoglycan (GAG) chain(s). The point of attachment is a serine residue to which the glycosaminoglycan is joined through a tetrasaccharide bridge. The chains are long, linear carbohydrate polymers that are negatively charged under physiological conditions, due to the occurrence of heparin sulfate and uronic acid groups. Proteoglycans form large complexes, both to other proteoglycans, to hyaluronan and to fibrous matrix proteins (such as collagen). They are also involved in binding cations (such as sodium, potassium and calcium) and water, and also regulating the movement of molecules through the matrix.

“Receptor” refers to a molecule or a polymeric structure in or on a cell that specifically recognizes and binds a compound acting as a molecular messenger (neurotransmitter, hormone, lymphokine, lectin, drug, etc.).

“Reperfusion injury” as used herein refers to damage to tissue caused when blood supply returns to the tissue after a period of ischemia. The absence of oxygen and nutrients from blood creates a condition in which the restoration of circulation results in inflammation and oxidative damage through the induction of oxidative stress rather than restoration of normal function.

The term “salt”, as used herein, denotes acidic and/or basic salts, formed with inorganic or organic acids and/or bases, preferably basic salts. While pharmaceutically acceptable salts are preferred, particularly when employing prostacyclin or an analog thereof of the invention as medicaments, other salts find utility, for example, in processing these prostacyclin analogs, or where non medicament-type uses are contemplated. Salts of these prostacyclin analogs may be prepared by art recognized techniques. Examples of such pharmaceutically acceptable salts include, but are not limited to, inorganic and organic acid addition safts, such as hydrochloride, sulphates, nitrates or phosphates and acetates, trifluoroacetates, propionates, succinates, benzoates, citrates, tartrates, fumarates, maleates, methane-sulfonates, isothionates, theophylline acetates, salicylates, respectively, or the like. Lower alkyl quaternary ammonium salts and the like are suitable, as well.

The term “syndecan 1” refers to the protein encoded by the human SDC1 gene or genes which are similar to the SDC1 gene. Within the meaning of syndecan 1 as mentioned here is both proteins encoded by full-length transcripts and shorter transcript variants. The protein is a transmembrane (type I) heparan sulphate proteoglycans and is a member of the syndecan proteoglycans family which comprises syndecan 1 to 4. The syndecan-1 protein functions as an integral membrane protein and participates in cell proliferation, cell migration and cell-matrix interactions via its receptor for extracellular matrix proteins.

The term “thrombocytopenia associated multi organ failure” (TAMOF) used herein will reflect any condition affecting critically ill patients related to development of multi-organ failure secondary to a pathological consumption of platelets resulting in thrombus formation in the microcirculation either due to thrombotic microangiopathic disease or secondary to disseminated intravascular coagulation or any other condition associated with a decline in platelet count and/or function.

“Trauma” as used herein is intended to mean any body wound or shock produced by sudden physical injury, as from accident, injury, or impact.

The terms “treatment” and “treating” as used herein refer to the management and care of a patient for the purpose of combating a condition, disease or disorder. The term is intended to include the full spectrum of treatments for a given condition from which the patient is suffering, such as administration of the prostacyclin or an analog thereof for the purpose of: ameliorating, alleviating or relieving symptoms or complications; delaying the progression of the condition, disease or disorder; curing or eliminating the condition, disease or disorder; and/or reducing the risk of or preventing the condition, disease or disorder, including preventing recurrence of the disease, wherein “preventing” or “prevention” is to be understood to refer to the management and care of a patient for the purpose of hindering the development of the condition, disease or disorder, and includes the administration of the pharmaceutical compositions to prevent the onset of symptoms or complications. The individual to be treated is preferably a mammal, in particular a human being. Treatment of animals, such as cattle, chickens, turkeys, ostriches, emu, ducks, horses, donkeys, mules, pigs, sheep, goats antelope, buffalo, llamas, cats, lions, tigers, dogs, bears, guinea pigs, hamsters, chinchillas, mink, ferrets, rodents, parrots, parakeets, peacocks, seals, sea lions, orcas, monkeys, chimpanzees, baboons, orangutans, gorillas, reptiles, and other zoo and livestock animals, is, however, also within the scope of the present invention. An individual to be treated according to the present invention can be of various ages and can be both female and male.

The term “unit dosage form” as used herein refers to physically discrete units suitable as unitary dosages for human and animal subjects, each unit containing a predetermined quantity of prostacyclin or an analog thereof, alone or in combination with other agents, calculated in an amount sufficient to produce the desired effect in association with a pharmaceutically acceptable diluent, carrier, or vehicle. The specifications for the unit dosage forms of the present invention depend on the particular compound employed which is prostacyclin or analog thereof and the effect to be achieved, as well as the pharmacodynamics associated with each compounds in the host.

### DETAILED DESCRIPTION OF THE INVENTION

### The Vascular Endothelium and its Glycocalyx

The endothelium is the thin layer of cells that lines the interior surface of blood vessels of the entire circulatory system, from the heart to the smallest capillary. The endothelium has many functions; one of them is to reduce turbulence of the flow of blood, allowing the fluid to be pumped faster. Further functions of the endothelium are exchange of solutes between tissue and blood and regulation of inflammatory responses and blood coagulation.

In healthy individuals, vascular endothelial cells are lined by a carbohydrate-rich layer called the glycocalyx. The vascular glycocalyx is linked to the endothelium through several “backbone” molecules, mainly proteoglycans and also glycoproteins. These form a network in which soluble molecules, either plasma- or endothelium-derived, are incorporated. On the side which is closest to the lumen of the blood vessels, the glycocalyx is formed by soluble plasma components which are linked to each other in a direct way or via soluble proteoglycans and/or glycosaminoglycans. The composition of the membrane-bound mesh of proteoglycans, glycoproteins, and glycosaminoglycans and the composition of associated plasma proteins and soluble glycosaminoglycans cannot be viewed as a static picture. A dynamic equilibrium exists between this layer of soluble components and the flowing blood, continuously affecting composition and thickness of the glycocalyx. In addition to being in equilibrium with the flowing blood, the glycocalyx suffers from enzymatic or shear-induced shedding. Enzymatic removal of any of its constituents dramatically affects glycocalyx properties, which exemplifies the importance of considering the synergetic interaction of all glycocalyx constituents as a whole.

Proteoglycans function as the “backbone” molecules of the glycocalyx. They consist of a core protein to which one or more glycosaminoglycan chains are linked. The core protein groups of syndecans (comprising syndecan 1-4) and glypicans (comprising GPC1-GPC6) have a firm connection to the cell membrane via a membrane-spanning domain (syndecans) or a glycosylphosphatidylinositol anchor (glypicans). Syndecan-1 (CD138) is the most prevalent type of syndecans in the endothelial glycocalyx. Syndecan functions as an integral membrane protein and participates in cell proliferation, cell migration and cell-matrix interactions via its receptor for extracellular matrix proteins. The variation is remarkable among the proteoglycan core proteins with regard to their size, number of attached glycosaminoglycan chains, and whether or not they are bound to the endothelial cell membrane. The glycosaminoglycan chains are typically heparan and chondroitin sulphates. Other proteoglycans, such as mimecan, perlecan, and biglycan, are secreted after their assembly and glycosaminoglycan chain modification. This leads to production of soluble proteoglycans, which reside in the glycocalyx or diffuse into the blood stream.

In addition to the above mentioned proteoglycans, the glycocalyx also comprises carbohydrates. A major component of the glycocalyx is hyaluronan, an anionic, nonsulfated glycosaminoglycan, which is often found as long polymers ranging from 5000 to 2 million Daltons. Hyaluronan contributes to tissue hydrodynamics, movement and proliferation of cells, and participates in a number of cell surface receptor interactions through the primary receptors CD44 and RHAMM.

Other components of the endothelial glycocalyx are a number of surface adhesion molecules on such as L-, E-, and P-selectin, β2-integrins, ICAM-1, ICAM-2 and VCAM-1. These are normally short compared to the thickness of the glycocalyx and therefore mostly masked by the glycocalyx in healthy individuals.

Medical Conditions

Degradation or shedding of the glycocalyx together with endothelial cell damage leads to aberration of endothelial barrier function and ensuing capillary leakage resulting in abnormal extravasation of blood components and accumulation of fluid in the extravascular space (hyperpermeability) and tissue edema, tissue dysfunction in part due to both impaired blood flow and ischemia which may lead to clinical conditions such as abdominal compartment syndrome, single/multi organ failure or systemic capillary leak syndrome.

Further, because degradation of glycocalyx and damage to the endothelial cells leads to exposure of surface adhesion molecules, these molecules are exposed for interaction and adhesion of platelet and leukocytes which can result in migration of polymorphonuclear neutrophils in the tissue.

Capillary leakage increases the need for intensive care treatment including pressor support (using drugs such as for example noradrenaline or dobutamine), ventilatory support, dialysis, treatment for septic complications and other treatments typically used in intensive care units (ICU) and prolongs the length of stay (LOS) for the patient in intensive care units or at the hospital, and in severe cases increases the mortality.

The risk of suffering from the capillary leakage is increased in elderly patients with co-morbidity such as diabetes, ischemic heart disease, atherosclerosis or other malignancies.

According to the present invention, prostacyclin or analogs thereof is used for prevention and/or treatment of capillary leakage.

In a specific embodiment of the present invention, the treatment using prostacyclin or analogs thereof mediates a protection of the endothelial cells and/or the endothelial glycocalyx.

Capillary leakage can cause intra-abdominal hypertension and abdominal compartment syndrome (ACS). ACS increases morbidity and mortality in a large variety of patients undergoing surgery. ACS occurs when the abdomen becomes subject to increased pressure (intra-abdominal hypertension) for example due to tissue edema. Increasing pressure reduces blood flow to abdominal organs and impairs pulmonary, cardiovascular, renal, and gastro-intestinal (GI) function, causing multiple organ dysfunction syndrome and death. Most treatment discussions have emphasized emergent surgical decompression, which involves opening the abdominal wall and abdominal fascia anterior in order to physically create more space for the abdominal viscera. Failure to provide emergent surgical decompression can lead to prolonged severe mesenteric ischemia as well as multiple organ hypoperfusion, which are conditions with high morbidity and mortality.

Medical management at an early stage has the potency to improve survival and prevents progression to full-blown ACS. It further reduces ICU and hospital length of stay, and results in reduced resource utilization.

In one embodiment of the present invention, the treatment as defined herein protects against or prevents intra-abdominal hypertension and/or development of abdominal compartment syndrome.

In one aspect the invention relates to the use of prostacyclin and analogs thereof for a treatment or prevention of capillary leakage which can lead to prevention or treatment of organ failure wherein organ failure is defined as altered organ function in a critically ill patient requiring medical intervention to achieve homeostasis. Organ failure includes as used herein single/multiple organ failure and thrombocytopenia associated multi organ failure, in at least one organ, such as in at least two, three, four or five organs. Organ failure is according to the present invention for example selected from the group of organ failures in the central nervous system, lungs, heart, gastrointestinal system, kidneys, liver and haematological systems.

Capillary leak syndrome (sometimes called systemic capillary leak syndrome or Clarkson syndrome, herein abbreviated CLS) is a medical condition characterized by a general hyperpermeability of the capillaries, which leads to a leakage of fluid from the blood to the interstitial fluid which can result in dangerously low blood pressure (hypotension), edema and multiple organ failure due to limited perfusion. The symptoms include low blood pressure (hypotension), hemoconcentration/hypovolemia, hypoalbuminemia without albuminuria and generalized edema. At the cellular level, the edema increases the diffusion distance between capillaries and tissue, resulting in increases in markers such as blood lactate and declining pH as indicators of organ ischemia and anaerobic metabolism due to impaired oxygen supply.

CLS can be described by two phases:
1. The capillary leak phase—Clinical features are abdominal pain, nausea, generalized edema and hypotension that may result in cardiopulmonary collapse. Acute renal failure is due to acute tubular necrosis consequent to hypovolemia and rhabdomyolysis.
2. Recruitment of the interstitial fluid. Intravascular overload with polyuria and pulmonary edema often occur. Edema may be more severe due to massive fluid supply in the initial phase. It is necessary to monitor the patient in order to switch to depletion treatment with diuretics or hemofiltration.

In one study of capillary leakage syndrome the mortality was reported to be 21% of the 57 cases.

In one embodiment of the present invention, the prostacyclin or an analog thereof as defined herein is used for prevention and/or treatment of capillary leakage syndrome.

Mechanisms of Endothelial Damage and Glycocalyx Degradation and Detection of Protection

The endothelial glycocalyx can be degenerated by enzymes acting on the different components of the glycocalyx. This degeneration results in shedding of components of the glycocalyx into the blood. Hyaluronan is degraded by a family of enzymes called hyaluronidases. In humans, there are at least seven types of hyaluronidase-like enzymes, several of which are tumor suppressors. The degradation products of hyaluronan, the oligosaccharides and very low-molecular-weight hyaluronan, exhibit pro-angiogenic properties. In addition, recent studies showed hyaluronan fragments, but not the native high-molecular mass of hyaluronan, can induce inflammatory responses in macrophages and dendritic cells in tissue injury. Hyaluronan degradation products transduce their inflammatory signal through toll-like receptor 2 (TLR2), TLR4 or both TLR2, and TLR4 in macrophages and dendritic cells. Syndecan extracellular domains (ectodomains) can be shed intact by proteolytic cleavage of their core proteins, yielding soluble proteoglycans that retain the binding properties of their cell surface precursors. Ectodomain shedding reduces the number of surface receptors, thus down-regulating signal transduction, converting membrane-bound syndecan 1 into a soluble effector competing for the same ligands. Shedding of syndecans and glypicans is accelerated by phorbol 12-myristate 13-acetate (PMA) activation of protein kinase C, and by ligand activation of the thrombin (G-protein-coupled) and EGF (protein tyrosine kinase) receptors. Mitogen-activated protein kinase (MAPK) signal transduction pathways also play an important role by mediating activation of several matrix metalloproteinases such as MT1-MMP (MMP-14) and MT3-MMP, MMP7 which degrade the proteoclycans in a context-dependent manner. Syndecan-1 and -4 ectodomains are found in acute dermal wound fluids, where they regulate growth factor activity. Overt endothelial damage is associated with shedding to thrombomodulin, and hence increased levels of soluble thrombomodulin in the blood.

In one embodiment of the present invention the treatments using prostacyclin or an analog thereof decreases the enzymatic cleavage of glycocalyx components, such as for example decreasing the enzymatic activity of one or more enzymes selected from heparanase, hyaluronidases, matrix metalloproteinases, and proteases released from neutrophil granulocytes such as but not limited to interstitial collagenase, matrix metalloproteiniase 8 (MMP-8), gelatinase B and matrix metalloproteinase 9 (MMP-9).

The glycocalyx can also be protected by an increased production of the components of the glycocalyx. In one embodiment of the present invention, the treatment as disclosed herein mediates increased expression and/or secretion of one or more of the components of the glycocalyx, for example, but not limited to, increased expression and/or secretion proteoglycans or carbohydrates mentioned herein.

The general state of glycocalyx and degradation can be measured using a number of different methods known in the art. Such methods include visualization techniques used in the art, for example microscopy such as light microscopy, electron microscopy, fluorescence microscopy and confocal laser scanning microscopy. Such visualization techniques may be combined with affinity labelling techniques such as lectin labelling or antibody labelling/immunohistochemistry using antibodies which bind to components of the glycocalyx such as for example heparan sulphate, syndecan-1 or hyaluronan.

Other methods for measuring the general state/degradation/protection of the endothelium and/or the glycocalyx can involve measuring the presence or level of markers of degradation in bodily fluids such as the blood, serum or plasma.

Such markers of endothelial and glycocalyx damage include soluble thrombomodulin, syndecan-1, glypican-1 and hyaluronan, which can be detected in increased levels in the blood after endothelial cell damage and degradation and shedding of the glycocalyx or components thereof.

These markers may include markers that are specific for the glycocalyx and/or markers that are specific for the endothelium.

Glycocalyx specific markers include, but are not limited to syndecan-1, glypican-1 and hyaluronan.

Syndecan-1 may be detected using a conventional ELISA method, such as Human Syndecan-1/CD138 ELISA Kit from CellSciences. Hyaluron may be detected by using conventional ELISA (USCN, kit no E90182Hu) and glypican-1 may be detected by using conventional ELISA (USCN, kit no E91032Hu).

In one embodiment of the present invention, the treatment as mentioned here is capable of preventing an increase in the plasma syndecan-1 levels to more than 2 to 5000 fold higher than normal, such as from 2 to 5 fold higher than normal, or such as from 5 to 20 fold higher than normal, or such from 30 to 50 fold higher than normal, or such as from 50 to 100 fold higher than normal, or such as from 100 to 200 fold higher than normal, or such as from 200 to 400 fold higher than normal, or such as from 400 to 1000 fold higher than normal, or such as from 1000 to 1500 fold higher than normal, or such as from 1500 to 2000 fold higher than normal, or such as from 2000 to 2500 fold higher than normal, or such as from 2500 to 3000 fold higher than normal, or such as from 3000 to 3500 fold higher than normal, or such as from 3500 to 4000 fold higher than normal, or such as from 4000 to 4500 fold higher than normal, or such as from 4500 to 5000 fold higher than normal, or such as more than 5000 fold higher than normal. Thus, in one embodiment of the present invention, the treatment as mentioned here is capable of preventing an increase in the plasma syndecan-1 levels to a level of more than 2 ng/ml to 600 ng/ml, such as more than a range of 2 ng/ml to 20 ng/ml, for example more than 2 ng/ml, such as more than 4.5 ng/ml, such as more than 10 ng/ml, such as more than 15 ng/ml, or such as more than a range of 20 ng/ml to 100 ng/ml, such as more than 30 ng/ml, such as more than 40 ng/ml, such as more than 50 ng/ml, such as more than 60 ng/ml, such as more than 70 ng/ml, such as more than 80 ng/ml, such as more than 90 ng/ml, or such as more than a range of 100 ng/ to 200 ng/ml, such as more than 100 ng/ml, such as more than 110 ng/ml, such as more than 120 ng/ml, such as more than 130 ng/ml, such as more than 140 ng/ml, such as more than 150 ng/ml, such as more than 160 ng/ml, such as more than 170 ng/ml, such as more than 180 ng/ml, such as more than 190 ng/ml, or such as more than a range of 200 ng/ to 300 ng/ml, such as more than 200 ng/ml, such as more than 210 ng/ml, such as more than 220 ng/ml, such as more than 230 ng/ml, such as more than 240 ng/ml, such as more than 250 ng/ml, such as more than 260 ng/ml, such as more than 270 ng/ml, such as more than 280 ng/ml, such as more than 290 ng/ml, or such as more than a range of 300 ng/ to 400 ng/ml, such as more than 300 ng/ml, such as more than 310 ng/ml, such as more than 320 ng/ml, such as more than 330 ng/ml, such as more than 340 ng/ml, such as more than 350 ng/ml, such as more than 360 ng/ml, such as more than 370 ng/ml, such as more than 380 ng/ml, such as more than 390 ng/ml, or such as more than a range of 400 ng/ to 500 ng/ml, such as more than 400 ng/ml, such as more than 410 ng/ml, such as more than 420 ng/ml, such as more than 430 ng/ml, such as more than 440 ng/ml, such as more than 450 ng/ml, such as more than 460 ng/ml, such as more than 470 ng/ml, such as more than 480 ng/ml, such as more than 490 ng/ml, or such as more than a range of 500 ng/ to 600 ng/ml, such as more than 500 ng/ml, such as more than 510 ng/ml, such as more than 520 ng/ml, such as more than 530 ng/ml, such as more than 540 ng/ml, such as more than 550 ng/ml, such as more than 560 ng/ml, such as more than 570 ng/ml, such as more than 580 ng/ml, such as more than 590 ng/ml, such as more than 595 ng/ml. It is thus an object of the present invention to maintain the syndecan-1 levels as close to normal levels as possible by administration of the compounds of the present invention. It follows that it is an object of the present invention to keep the syndecan-1 level in the vicinity of 120 ng/ml+/−5 ng/ml, such as 120 ng/ml+/−10 ng/ml, such as 120 ng/ml+/−15 ng/ml, such as 120 ng/ml+/−20 ng/ml, such as 120 ng/ml+/−30 ng/ml, such as 120 ng/ml+/−40 ng/ml, such as 120 ng/ml+/−50 ng/ml, such as 120 ng/ml+/−60 ng/ml, such as 120 ng/ml+/−70 ng/ml, such as 120 ng/ml+/−80 ng/ml, such as 120 ng/ml+/−90 ng/ml.

In one embodiment of the present invention, the treatment as mentioned here is capable of preventing an increase in the plasma glypican-1 levels compared to normal levels, such as more than 2 fold higher than normal, such as more than 10 fold higher than normal, such as more than 20 fold higher than normal, such as more than 30 fold higher than normal, such as more than 40 fold higher than normal, such as more than 50 fold higher than normal, such as more than 60 fold higher than normal, such as more than 70 fold higher than normal, such as more than 80 fold higher than normal, such as more than 90 fold higher than normal, such as more than 100 fold higher than normal, such as more than 110 fold higher than normal, such as more than 120 fold higher than normal, such as more than 130 fold higher than normal, such as more than 140 fold higher than normal, such as more than 150 fold higher than normal. Thus, in one embodiment of the present invention, the treatment as mentioned here is capable of preventing an increase in the plasma glypican-1 levels to more than 0.10 ng/ml, such as more than 0.20 ng/ml, such as more than 0.30 ng/ml, such as more than 0.40 ng/ml, such as more than 0.50 ng/ml, such as more than 0.60 ng/ml, such as more than 0.70 ng/ml, such as more than 0.80 ng/ml, such as more than 0.90 ng/ml, such as more than 1 ng/ml, such as more than 2 ng/ml, such as more than 5 ng/ml, such as more than 7.5 ng/ml, such as more than 10 ng/ml, such as more than 12.5 ng/ml, such as more than 15 ng/ml, such as more than 20 ng/ml.

The effects of the treatment using prostacyclin or an analog thereof can also be monitored by measuring levels of markers indicating endothelial cell activation or endothelial damage, i.e. endothelial markers. Thus, such a method can include the measuring in a bodily fluid (such as for example plasma, serum and blood) of the levels of adrenaline, noradrenaline, ICAM-1, E-selectin, Soluble fms-like tyrosine kinase-1 (also called sFlt-1 or sVEGF R1), sVE-cadherin, angiopoietin 1 (Ang-1), angiopoietin 2 (Ang-2), soluble thrombomodulin (sTM), soluble endothelial protein C receptor (sEPCR), protein C (PC), activated protein C (APC), Antithrombin III (AT) (Antithrombin III), tissue factor pathway inhibitor (TFPI), von Willebrand factor (vWF), tissue-type plasminogen activator (tPA), Factor XIII, histon-complexed DNA fragments, high-mobility group protein B1 (HMGB1), d-dimer, interleukin-6 (IL-6) and terminal complement complex sC5B9).

Preferably the endothelial markers include, but are not limited to, ICAM-1, E-selectin, Soluble fms-like tyrosine kinase-1 (also called sFlt-1 or sVEGF R1), sVE-cadherin, angiopoietin 2 (Ang-2), soluble thrombomodulin (sTM), soluble endothelial protein C receptor (sEPCR), protein C (PC), activated protein C (APC), Antithrombin III (AT) (Antithrombin III), tissue factor pathway inhibitor (TFPI), von Willebrand factor (vWF), and tissue-type plasminogen activator (tPA). More preferably the markers are E-selectin, soluble thrombomodulin (sTM), protein C (PC), Antithrombin III (AT) and (Antithrombin III),

In the clinic, prevention of capillary leakage may result in a reduction in the risk or decrease or of one or more of the following factors:
a. need for fluid administration perioperatively and/or post-surgery, or the volume of fluid administered perioperatively and/or post-surgery,
b. weight gain 12 hours, 24 hours and 48 hours post-surgery compared to pre-surgery,
c. incidence of abdominal compartment syndrome post-surgery,
d. need for supportive therapy such as intensive care therapy such as, but not limted to, pressor support (drugs such as noradrenaline, dobutamine), ventilatory support, dialysis and treatment for septic complications,
e. occurrence of single/multiple organ failures selected from the group of organ failures in the central nervous system, lungs, heart, gastrointestinal system, kidneys, liver and haematological systems, and
f. coagulopathy (impairment in coagulation which can cause hypocoagulability or hypercoagulability).

In one embodiment of the present invention, the general state of/protection/degradation of glycocalyx and endothelial cells may be monitored by a comparison of the measured levels of the above mentioned markers or clinical factors measured in individuals who are treated as defined herein and comparing said measured factors to measured factors in individuals which are subject to the same type of surgery, but not treated with prostacyclin or an analog thereof as defined herein. In another embodiment of the present invention, the general state of/protection/degradation of glycocalyx and endothelial cells may be measured by a comparison of the level of the above mentioned markers or factors measured in an individual prior to treatment, during treatment or after treatment as defined herein.

In one embodiment of the present invention, the treatment with prostacyclin or an analog thereof leads to discrete or minimal effects on the hemostatic system such as a significant inhibition of platelet aggregation (for instance measured by electrical impedance aggregometry) or coagulation of blood (for instance measured by thrombelastography (TEG)). The present invention thus provides a treatment, which can allow for prevention or treatment of capillary leakage during surgery and a recovery period after surgery wherein the healing of tissue proceeds via normal hemostasis involving platelet (thrombocyte) aggregation.

Viscoelastical Citrated Whole Blood Haemostasis Assay: Thrombelastography (TEG) or Thrombelastometry (ROTEM)

The TEG in vitro assay is suitable for determining important parameters in the platelet aggregation, clotting activity and clot strength. The TEG system's approach to monitoring patient haemostasis is based on the premise that the end result of the haemostatic process is the clot. The clot's physical properties determine whether the patient will have normal haemostasis, or will be at increased risk for haemorrhage or thrombosis (Salooja et al. 2001).

The TEG analyzer uses a small whole blood sample in a rotating cup and a pin suspended in the blood by a torsion wire, which is monitored for motion. To speed up the clot formation, a standardized amount of an activator of coagulation (e.g. Kaolin, tissue factor) may be added to the cup just before the pin is placed in the cup. The torque of the rotating cup is transmitted to the immersed pin only after fibrin and/or fibrin-platelet bonding has linked the cup and pin together. The strength and rate of these bonds affect the magnitude of the pin motion such that strong clots move the pin directly in phase with cup motion. Thus, the TEG technology documents the interaction of platelets with the protein coagulation cascade from the time of placing the blood in the analyzer until initial fibrin formation, clot rate strengthening and fibrin-platelet bonding via GPIIb/IIIa, through eventual clot lysis. The TEG R parameter reflects the initiation phase, reaction time, from start of coagulation until the first fibrin band is formed; the Angle (α) represents the increase in clot strength, clot kinetics, correlating with the thrombin generation. The maximal amplitude (MA) parameter reflects maximal clot strength i.e. the maximal elastic modus of the clot. Ly30 demonstrate the proportion of the clot that is dissolved 30 min after MA is reached, reflecting fibrinolysis.

The clot strength, stability and changes herein may be measured as increases in relative clot strength by the TEG (Thrombelastography) measurable parameter MA and clot stability by the TEG derivable parameter Lysis AUC. The maximal amplitude (MA) parameter reflects maximal clot strength i.e. the maximal elastic modus of the clot. The area under the lysis curve, i.e. area under the curve from MA is obtained (Lysis AUC) reflects degree of fibrinolysis. Both clot strength and stability may be measured, or one parameter only may be followed during a procedure such as either the clot stability or the clot strength.

Normal values for the above mentioned parameters measured by TEG in a citrated whole blood sample activated by kaolin are

a) R between 3.0 to 8.0 minutes.

b) Angle between 55° and 78°.

c) MA between 51 mm to 69 mm.

The TEG system has been recognized as a uniquely useful tool and has been used extensively in the management of haemostasis during major surgical interventions such as liver transplantations (Kang et al 1985) and cardiovascular procedures as well as obstetrics, trauma, neurosurgery, management of deep vein thrombosis, and the monitoring and differentiation among platelet GPIIb/IIIa antagonists (Di Benedetto 2003).

Thus in one embodiment of the present invention, the treatment with prostacyclin or an analog thereof has minimal effects on the hemostatic system as measured by TEG. In other words it is an aspect of the present invention to administer the compounds of the present invention in a manner that has little to no effect on the TEG values for said individual after treatment as compared to the values measured prior to treatment.

Therefore, for a person with TEG values within the normal ranges in one embodiment of the present invention, the treatment with prostacyclin or an analog thereof has minimal effects on the hemostatic system as measured by TEG, wherein the TEG values in a citrated blood sample activated by kaolin are a) R between 3.0 to 8.0 minutes, b) Angle between 55° and 78°, and c) MA between 51 mm to 69 mm.

Likewise, in one embodiment of the present invention, the treatment with prostacyclin or an analog thereof has minimal effects on the hemostatic system as measured by Multiplate whole blood aggregometry.

Multiplate whole blood aggregometry is a platelet function test, (Multiplate®, Dynabyte Medical, Munich, Germany). This test is based on multiple electrode platelet aggregometry (MEA), which measures platelet aggregation in whole blood (WB) after stimulation with selective platelet agonists such as trombin-activated peptide (TRAP), adenosine diphosphate (ADP), arachidonic acid (ASPI) and collagen (COL). The increase of impedance by the attachment of platelets onto the Multiplate sensors is transformed to arbitrary aggregation units (AU) and plotted against time. The aggregation can then be measured in AUC*min (area under the curve*min) determined by the plot of units against time. Additionally, the aggregation can also be expressed in units (U) (1 unit=10 AU*min). Multiplate thereby allows analyzing the effect of compounds on platelet aggregation. In heparin-treated whole blood from healthy humans, the mean values of AUC and the values for the 95% confidence interval (CI) are typically:

ADP: 84 AUC*min, 47-121 95% Cl.

ASPI: 96 AUC*min, 59-133 95% Cl.

TRAP: 114 AUC*min, 66-163 95% Cl.

Or as stated by the producers of the system: the median values of AUC and the values for the 90% confidence interval (CI) are typically:

ADP: 83 AUC*min, 55-117 95% Cl.

ASPI: 106 AUC*min, 79-141 95% Cl.

TRAP: 121 AUC*min, 92-151 95% Cl.

Thus in one embodiment of the present invention, the treatment with prostacyclin or an analog thereof has minimal effects on the hemostatic system as measured by multiplate whole blood aggregometry, and the aggregation units (AU) measured after stimulation with ADP, ASPI, TRAP and/or COL in whole blood samples obtained during or after treatment are within the ranges of healthy humans. Further, according to the present invention, treatment as defined herein leads to aggregation units measured on sample of whole blood obtained during or after treatment in the range of 40 to 200, such as aggregation units during or after treatment in the range of 40 to 50, such as aggregation units during or after treatment in the range of 50 to 60, such as aggregation units during or after treatment in the range of 60 to 70, such as aggregation units during or after treatment in the range of 70 to 80, such as aggregation units during or after treatment in the range of 80 to 90, such as aggregation units during or after treatment in the range of 90 to 100, such as aggregation units during or after treatment in the range of 100 to 110, such as aggregation units during or after treatment in the range of 110 to 120, such as aggregation units during or after treatment in the range of 120 to 130, such as aggregation units during or after treatment in the range of 130 to 140, such as aggregation units during or after treatment in the range of 140 to 150, such as aggregation units during or after treatment in the range of 150 to 160, such as aggregation units during or after treatment in the range of 160 to 170, such as aggregation units during or after treatment in the range of 170 to 180, such as aggregation units during or after treatment in the range of 180 to 190, such as aggregation units during or after treatment in the range of 190 to 200.

Prostacyclin and Prostacyclin Analogs

Prostacyclin, a metabolite of arachidonic acid, is a naturally occurring prostaglandin with potent vasodilatory activity and inhibitory activity of platelet aggregation, released by healthy endothelial cells. Prostacyclin can performs its function through a paracrine signalling cascade that involves the prostacyclin receptor (IP receptor) which is G protein-coupled receptor located on platelets and endothelial cells. In a conventional clinical setting, prostacyclin and analogs thereof that are used for treatment, has 2 major pharmacological actions: (1) direct vasodilation of pulmonary and systemic arterial vascular beds, (2) inhibition of platelet aggregation/coagulation of the blood.

Binding of prostacyclin and analogs thereof to endothelial prostacyclin receptors can lead to a rise in cytosolic cAMP and Protein Kinase A activation. This can further lead to smooth muscle relaxation and vasodilatation with improved microvascular perfusion and “cytoprotection” through stabilization of lysozomal and cell membranes with reduced inflammation (Zardi et al 2005; Zardi et al 2007).

The antiaggregatory effect of prostacyclin and analogs thereof is mediated by activation of the prostacyclin receptor (Gas protein-coupled receptor) upon prostacyclin analog binding. This activation signals adenylyl cyclase to produce cAMP, which in turn activates Protein Kinase A to decrease free intracellular calcium concentrations. The rise in cAMP directly inhibits platelet activation (secretion and aggregation) and counteracts increases in cytosolic calcium resulting from platelet activation by agonists such as thrombin, adenosine diphosphate (ADP), thromboxane A2 (TXA2), platelet activating factor (PAF), collagen and serotonin (5-HT) (Bihari et al, 1988; Schereen et al, 1997; Xing et al 2008).

The inventors of the present invention have surprisingly found that prostacyclin or analogs thereof given in low doses during surgery has a positive effect on prevention and treatment of capillary leakage. When administered in such low doses during surgery, prostacyclin and an analog thereof have minimal effect on the platelet aggregation and coagulation.

Low doses of prostacyclin or an analog thereof as mentioned herein can further be capable of mediating a discrete vasodilation of the microcirculation.

Thus in one embodiment of the present invention, the treatment as defined herein is capable of protecting the glycocalyx while minimizing bleeding, mediating a discrete vasodilation and protecting cell membranes, such as for example endothelial cells, cell membranes of the endothelial cells and the glycocalyx.

According to the present invention, a compound which is prostacyclin or an analog thereof is administered to an individual for prevention or treatment of capillary leakage. Such compounds of the present invention are selected from the group, but not limited to: PGI2, PGX, prostacyclin (Epoprostenol) or variants thereof, such as beraprost sodium, epoprostenol sodium, iloprost, iloprost in combination with bosentan, iloprost in combination with sildenafil citrate, treprostinil, pegylated treprostinil, treprostinil diethanolamine and treprostinil sodium or derivatives thereof. Further compounds which are prostacyclin analogs are 2-{4-[(5,6-diphenylpyrazin-2-yl)(isopropyl)amino]-butoxy}-N-(methylsulfonyl)acetamide, {4-[(5,6-diphenylpyrazin-2-yl)(isopropyl)amino]-butoxy}acetic acid, 8-[1,4,5-triphenyl-1H-imidazol-2-yl-oxy]octanoic acid, isocarbacyclin, cicaprost, [4-[2-(1,1-Diphenylethylsulfanyl)-ethyl]-3,4-dihydro-2H-benzo[1,4]oxazin-8-yloxy]-acetic acid N-Methyl-d-glucamine, 7,8-dihydro-5-(2-(1-phenyl-1-pyrid-3-yl-methiminoxy)-ethyl)-a-naphthyloxyacetic acid, (5-(2-diphenylmethyl aminocarboxy)-ethyl)-a-naphthyloxyaceticacid, 2-[3-[2-(4,5-diphenyl-2-oxazolyl)ethyl]-phenoxy]acetic acid, [3-[4-(4,5-diphenyl-2-oxazolyl)-5-oxazolyl]phenoxy]acetic acid, bosentan, 17[alpha],20-dimethyl-[DELTA]6,6a-6a-carba PGI1, and 15-deoxy-16[alpha]-hydroxy-16[beta],20-dimethyl-[DELTA]6,6a-6a-carba PGI1 and salts, hydrates, solvates and derivatives thereof.

The prostacyclin analogs epoprostenol sodium and iloprost are equipotent and particularly useful for treatment as described herein. (These are marketed for intravenous administration under the trade names Flolan and Ilomedin). In a preferred embodiment of the present invention, the prostacyclin analog is epoprostenol sodium or iloprost.

In another preferred embodiment the prostacyclin analog has a half time of less than 4 hours (such as treprostinil), preferably less than 1 hours (such as beraprost (35-40 min)), more preferably less than ½ hour (such as Iloprost (20-30 min)), preferably less than 5 min (such as epoprostenol (0.5-3 min)).

Surgery

The types of surgery in which the treatment should be applied includes gastroenterology procedures, thoracic procedures, urology or gynecology procedures, plastic/cosmetic/reconstructive procedures and orthopedic procedures performed due to infections, non-infectious antigens, inflammation, burn/erosions, organ/tissue destruction/degeneration/damage/fracture, haemorrhage, intoxication, and/or malignancy in/of organs/tissues of the body i.e., lungs, heart, stomach, duodenum, ileum, jejunum, colon, sigmoideum, rectum, liver, pancreas, gallbladder, kidneys, spleen, bladder, uterus, prostate, hip, knees, back.

A non-extensive list of procedures within each organ system includes:

Gastroenterology Procedures:

Abscess Incision and Drainage

Adrenalectomy

Antrectomy

Cholecystectomy

Colon resection

Colostomy

Diverticulectomy

Fistulotomy

Fundectomy

Gastrectomy

Gastrostomy

Gastroduodenostomy

Gastroesophageal Reflux Surgery

Hemicorporectomy

Hemilaminectomy

Hepatectomy

Heal Conduit Surgery

Ileostomy

Intestinal Obstruction Repair

Intussusception Reduction

Laparotomy, Laporatomy exploratory

Pancreaticoduodenectomy (Whipple operation)

Rectal Resection

Sigmoidostomy

Small and large Bowel Resection

Sphincterotomy

Splenectomy

Thrombectomy

Vasectomy

Vertical banded gastroplasty

Thoracic Procedures:

Pneumotomy

Pneumonectomy

Thoracotomy

Orthopedic Procedures:

Acetabuloplasty

Amputation

Arthrodesis

Arthroplasty

Fracture Repair

Hip Osteotomy

Hip Replacement

Hip Revision Surgery

Knee Osteotomy

Knee Replacement

Knee Revision Surgery

Pelvic surgery

Uro-/Gynecology Procedures:

Cystectomy

Hysterectomy

Nephrectomy

Prostatectomy

Pyloroplasty

Ureterosigmoidostomy

Plastic-/Cosmetic-/Reconstructive-Procedures:

Burn surgery

Necrotomy

Reconstructive surgery

Trauma Surgery

In a particular embodiment of the present invention, the treatments as described herein are used during surgery that does not involve a significant risk of ischemia or reperfusion injury of tissues, for instance such as unstable anginal ischemia or myocardial infarction.

In another embodiment of the present invention, the treatments as described herein are used during surgery that is not related to cardiovascular diseases or atherosclerosis such as angioplasty, bypass operations, heart failure operations or insertion of catheters, stents or grafts.

In one embodiment of the present invention, the treatments as described herein are used during surgery that does not involve trauma such as for example head injury, brain trauma or skeletal muscle trauma, i.e. neurotrauma is not an object of the present invention.

In another embodiment of the present invention, the treatments as described herein are used during surgery that does not involve a significant risk of pulmonary hypertension.

In another embodiment of the present invention, the treatments as described herein are used during surgery that does not involve transplantation.

Administration and Dose

According to the present invention, prostacyclin or an analog thereof is administered during surgery (periooperative administration). Thus in one embodiment of the present invention, prostacyclin or an analog thereof is administered pre-operatively, intraoperatively and/or postoperatively.

In a preferred embodiment of the present invention, prostacyclin or an analog thereof is administered intraoperatively during surgery, which is any time-point in the interval starting from induction of anaesthesia in the patient to the finishing of the last suturing or stampling of the surgical operation.

In another preferred embodiment of the present invention, prostacyclin or an analog thereof is administered intraoperatively during surgery, which is any time-point in the interval starting from induction of anaesthesia in the patient to the finishing of the last suturing or stampling of the surgical operation and post-operatively after the surgery. The post-operative administration may last for any length of time, preferably for at least 1 hour, such as 2 hours, 3 hours, 4 hours, 6 hours, 12 hours, 24 hours, 36 hours, 48 hours, 60 hours, 72 hours, 84 hours or more. It is preferred that the post-operative administration lasts 72 hours.

In another embodiment of the present invention, prostacyclin or an analog thereof is administered intraoperatively at any time-point during the range of time wherein invasive procedures are performed on the body, such as from the first incision to the last incision of the surgery.

In yet an embodiment of the present invention, prostacyclin or an analog thereof is administered intraoperatively at any time-point during the range of time wherein invasive procedures are performed on the body, such as from the first incision to at least 72 hours after the surgery.

In one embodiment of the present invention, the treatment of the present invention corresponds to the length of the period of surgery. Thus, according to the present invention, the treatment is performed in an interval of 15 min to 360 min, for example an interval of 15 to 60 min, such as 15 min to 30 min, or such as 30 min to 45 min, or such as 45 min to 60 min, or for example an interval of 60 min to 120 min, such as 60 min to 75 min, or such as 75 min to 90 min, or such as 90 min to 105 min, or such as 105 min to 120 min, such as such as 120 min to 135 min, or such as 135 min to 150 min, or such as 150 min to 165 min, or such as 165 min to 180, or for example an interval of 180 min to 240 min, such as 180 min to 195 min, or such as 195 min to 210 min, or such as 210 min to 225 min, or such as 225 min to 240, or for example an interval of 240 min to 300 min such as 240 min to 255 min, or such as 255 min to 270 min, or such as 270 min to 300 min, or for example an interval of 300 min to 360 min such as 300 min to 315 min, or such as 315 min to 330 min, or such as 330 min to 345 min, or such as 345 min to 360 min.

It is also an object of the present invention to administer the treatment of the present invention post-operatively, thus the administration of the treatment may continue after the surgery for more than about 30 min, such as about 1 hour, such as 2 hours, such as 3 hours, such as 4 hours, such as 5 hours, such as 6 hours, such as 8 hours, such as 10 hours, such as 12 hours, such 12 14 hours, such as 16 hours, such as 18 hours, such as 20 hours, such as 22 hours, such as 24 hours, such as 26 hours, such as 28 hours, such as 30 hours, such as 32 hours, such as 26 hours, such as 40 hours, such as 44 hours, such as 48 hours, such as 52 hours, such as 56 hours, such as 60 hours, such as 64 hours, such as 68 hours, such as 72 hours, such as 76 hours, such as 80 hours, such as 86 hours, such as 92 hours, such as 100 hours.

The treatment may thus be administered both during and after surgery. Preferably the treatment is administered during surgery and for up to about 72 hours after the surgery.

Administration of prostacyclin or an analog thereof and/or compositions of the present invention are to be given to a subject resulting in a systemic concentration of the prostacyclin or analog thereof. Methods of administration include enteral, such as oral, sublingual, gastric or rectal and/or parenterally, that is by intravenous, intraarterial, intramuscular, subcutaneous, intranasal, intrapulmonary, intrarectal, intravaginal or intraperitoneal administration. The subcutaneous and intravenous forms of parenteral administration are generally preferred. Appropriate dosage forms for such administration may be prepared by conventional techniques. Prostacyclin or an analog thereof may also be administered by inhalation that is by intranasal and oral inhalation administration. Appropriate dosage forms for such administration, such as an aerosol formulation or a metered dose inhaler, may be prepared by conventional techniques.

According to a highly preferred embodiment of the present invention prostacyclin or an analog thereof is administered by continuous parental infusion, preferably by continuous intra venous (i.v.) infusion.

Low doses of prostacyclin or an analog thereof have been found by the inventors of the present invention to have a beneficial effect on the treatment or prevention of capillary leakage. Further, the doses of prostacyclin or an analog thereof used in the present invention may not cause significant changes of the hemostatic system such as a significant inhibition of platelet aggregation.

In another embodiment of the present invention, the treatment using prostacyclin or an analog thereof involves a hemodynamic profile of the patient which is normal or close to normal, said hemodynamic profile being measured by for instance blood pressure monitoring (which may monitored by invasive cannulation), and/or electrogradiography (ECG) and/or oxygen saturation in the blood. An estimate of normal hemodynamic profile is determined by monitoring the patient prior to surgery.

In treatment methods of the present invention, prostacyclin and analogs thereof are administered in doses of 0.5 ng/kg/min to 4.0 ng/kg/min, for instance in the interval between 0.5 ng/kg/min to 1.0 ng/kg/min, such as 0.5 ng/kg/min to 0.75 ng/kg/min, or such as 0.75 ng/kg/min to 1.0 ng/kg/min, or for instance in the interval between 1.0 ng/kg/min to 3.0 ng/kg/min, such as 1.0 ng/kg/min to 1.25 ng/kg/min, or such as 1.25 ng/kg/min to 1.50 ng/kg/min, or such as 1.50 ng/kg/min to 1.75 ng/kg/min, or such as 1.75 ng/kg/min to 2.0 ng/kg/min, or such as 2.0 ng/kg/min to 2.25 ng/kg/min, or such as 2.25 ng/kg/min to 2.5 ng/kg/min, or such as 2.5 ng/kg/min to 2.75 ng/kg/min, or such as 2.75 ng/kg/min to 3.0 ng/kg/min, or for instance in the interval between 3.0 ng/kg/min to 4.0 ng/kg/min such as 3.0 ng/kg/min to 3.25 ng/kg/min, or such as 3.25 ng/kg/min to 3.5 ng/kg/min, or such as 3.5 ng/kg/min to 3.75 ng/kg/min, or such as 3.75 ng/kg/min to 4.0 ng/kg/min.

In another embodiment the compound capable of modulating/preserving endothelial integrity particularly prostacyclin (PGI2), prostacyclin (PGX), or variants thereof, most preferably iloprost or flolan, the dose administered will for parenteral routes, in particular intravenous, intramuscular, and/or subcutaneous routes, in a single or repeated bolus dose corresponding to maintaining a systemic concentration of about 0.5-4.0 ng/kg for a period of time, such as for 10 minutes, more preferably 15 minutes, more preferably 30 minutes, such as 60 minutes, 90 minutes or 120 minutes during intraoperative conditions. More preferably the systemic concentration is about 0.5-2.0 ng/kg for the period of time. The systemic concentration may be adjusted according to the response observed in the individual treated and may be adjusted to 0.5 ng/kg, 1.0 ng/kg, 1.5 ng/kg, 2.0 ng/kg, 2.5 ng/kg, 3.0 ng/kg, 3.5 ng/kg or 4.0 ng/kg such as by increasing or decreasing the dosage administered every 15 minutes or so.

It is further envisioned to maintain a systemic concentration of about 0.5-4.0 ng/kg for a period of time that includes the intraoperative and post-operative period for a period of time post-operatively of about 12 hours, such as 24 hours, such as 36 hours, such as 48 hours, such as 60 hours, such as 72 hours or more.

Although some of the compounds normally are known to have adverse effect on bleeding, it has been found that when administered in the low dosages herein then the desired effect on the endothelium and/or the glycocalyx is obtained without the adverse effect on bleeding.

The compound may be administered by a one or more bolus injections, and accordingly, the bolus injection may be given once, twice or several times, for instance, in keeping with the dosage administered the bolus injection may be given every 5 min (minutes), such as every 10 min, such as every 15 min, such as every 20 min, such as every 25 min, such as every 30 min, such as every 35 min, such as every 40 min, such as every 45 min, such as every 50 min, such as every 55 min, such as every 60 min such as every 70 min, such as every 80 min, such as every 90 min, such as every 100 min, such as every 110 min such as every 120 min or more. For example, the bolus dosage may be administered in the appropriate intervals from the time of trauma to the subject and until a treatment facility such as a hospital or other is reached. Particularly it is of interest to administer the compounds of the present invention intraoperatively as bolus injections with any of the above frequencies. In a further embodiment it is of interest to administer the compounds of the present invention both during surgery and post-operatively for up to at least 72 hours after the surgery, by administering bolus doses repeatedly with any of the above given intervals.

Thus the compounds of the present invention may be administered as a single bolus dose or as repeated doses.

The compounds of the present invention may be formulated for parenteral administration (e.g., by injection, for example bolus injection or continuous infusion) and may be presented in unit dose form in ampoules, pre-filled syringes, small volume infusion or in multi-dose containers with an added preservative.

Normally the dose should be capable of preventing or lessening the severity or spread of the condition or indication being treated. The exact dose selected from the ranges mentioned herein will depend on the circumstances, such as the condition being treated, the administration schedule, whether the prostacyclin or an analog thereof is administered alone or in conjunction with another therapeutic agent, the plasma half-life of prostacyclin or analog thereof and the general health of the subject.

Second Active Ingredients

The treatment of the present invention may involve a use of a second active ingredient for the protection of glycocalyx and prevention/treatment of the complications occurring due to a degradation of the glycocalyx.

Modulation of the sympathoadrenal system is capable of mediating a balanced hemostatic response, which may result in a decreased bleeding associated with surgery. Accordingly, prostacyclin or an analog thereof may be administered together with one or more antagonists and/or agonists of adrenergic receptors. Thus in one embodiment of the present invention, prostacyclin or an analog thereof is administered in combination with an alpha-1 (α1) adrenergic receptor agonist such as Methoxamine, Methylnorepinephrine, Oxymetazoline and Phenylephrine.

In another embodiment of the present invention, prostacyclin or an analog thereof is administered in combination with a alpha-2 (α2) adrenergic receptor agonist such as Clonidine, Guanfacine, Guanabenz, Guanoxabenz, Guanethidine, Xylazine, Methyldopa and Fadolmidine.

In yet another embodiment of the present invention, prostacyclin or an analog thereof is administered in combination with a undetermined a adrenergic receptor agonist such as amidephrine, amitraz, anisodamine, apraclonidine, brimonidine, cirazoline, detomidine, dexmedetomidine, epinephrine, ergotamine, etilefrine, indanidine, lofexidine, medetomidine, mephentermine, metaraminol, methoxamine, midodrine, mivazerol, naphazoline, norepinephrine, norfenefrine, octopamine, oxymetazoline, phenylpropanolamine, rilmenidine, romifidine, synephrine, talipexole and tizanidine.

In yet another embodiment of the present invention, prostacyclin or an analog thereof is administered in combination with beta-1 adrenergic receptor agonists such as Dobutamine, Isoproterenol, Xamoterol and epinephrine.

In yet another embodiment of the present invention, prostacyclin or an analog thereof is administered in combination with a beta-2 adrenergic receptor agonist such as salbutamol, Fenoterol, Formoterol, Isoproterenol, Metaproterenol, Salmeterol, Terbutaline, Clenbuterol, Isoetarine, pirbuterol, procaterol, ritodrine and epinephrine.

In yet another embodiment of the present invention, prostacyclin or an analog thereof is administered in combination with an undetermined beta adrenergic receptor agonist such as arbutamine, Befunolol, bromoacetylalprenololmenthane, broxaterol, cimaterol, cirazoline, denopamine, dopexamine, etilefrine, hexoprenaline, higenamine, isoxsuprine, mabuterol, methoxyphenamine, nylidrin, oxyfedrine, prenalterol, ractopamine, reproterol, rimiterol, tretoquinol, tulobuterol, zilpaterol and zinterol.

In yet another embodiment of the present invention, prostacyclin or an analog thereof is administered in combination with adrenerge receptor antagonists such as but not limited to: alpha-1 (α₁) adrenergic receptor antagonists such as Alfuzosin, Arotinolol, Carvedilol, Doxazosin, Indoramin, Labetalol, Moxisylyte, Phenoxybenzamine, Phentolamine, Prazosin, Silodosin, Tamsulosin, Terazosin, Tolazoline, Trimazosin, and/or alpha-2 (α₂) adrenergic receptor antagonists such as Atipamezole, Cirazoline, Efaroxan, Idazoxan, Mianserin, Mirtazapine, Napitane, Phenoxybenzamine, Phentolamine, Rauwolscine, Setiptiline, Tolazoline, Yohimbine and/or beta-1 adrenergic receptor antagonists such as Acebutolol, Atenolol, Betaxolol, Bisoprolol, Esmolol, Metoprolol, Nebivolol, and/or beta-2 adrenergic receptor antagonists such as Butaxamine, ICI-118,551, and/or non-selective beta-blockers such as Bucindolol, Alprenolol, Carteolol, Carvedilol (has additional α-blocking activity), Labetalol (has additional α-blocking activity), Nadolol, Penbutolol, Pindolol, Propranolol, Sotalol, Timolol and/or Beta-3 adrenergic receptor antagonists such as SR 59230A (has additional α-blocking activity) and/or other modulators of the sympathoadrenal system that can be combined with prostacyclin such as Levosimendan, Hydrocortizone and/or Arginine vasopressin.

In yet another embodiment of the present invention, prostacyclin or an analog thereof is administered in combination with other modulators of the sympathoadrenal system such as mentioned by Dunser: J Int Care Med 2009; 24:293-316, for example such as Levosimendan, Hydrocortizone and Arginine vasopressin.

In a preferred embodiment of the present invention a second active ingredient is epinephrine.

Second active ingredients for protection of the glycocalyx may also be administered in combination with prostacyclin or an analog thereof. In another embodiment of the present invention prostacyclin or an analog thereof is administered in combination with Antithrombin III (AT), hydrocortisone, glucocorticoids, N-acetylcysteine, plasma, valproate or albumin.

Pharmaceutical Composition

The present invention relates to a pharmaceutical composition for treatment as defined herein, which comprises prostacyclin or an analog thereof and one or more pharmaceutically acceptable carriers or excipients. Such pharmaceutically acceptable carrier or excipients as well as suitable pharmaceutical formulation methods are well known in the art (see for example Remington's Pharmaceutical Sciences, 18^{th} ed., Mack Publishing Company, Easton, Pa. (1990).

In a preferred embodiment prostacyclin or an analog thereof is prepared in a parenteral composition. Such methods for preparing parenterally administrable compositions will also be known or apparent to those skilled in the art and are described in more detail in, for example, Remington's Pharmaceutical Sciences, 18^{th} ed., Mack Publishing Company, Easton, Pa. (1990). As used herein, the term “pharmaceutical acceptable” means carriers or excipients that does not cause any untoward effects in subjects to whom it is administered.

According to the present invention, prostacyclin or an analog thereof may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

The compositions for parenteral administration comprise prostacyclin or an analog thereof in combination with, preferably dissolved in, a pharmaceutically acceptable carrier, preferably an aqueous carrier. A variety of aqueous carriers may be used, such as water, buffered water, saline e.g. such as 0.7%, 0.8%, 0.9% or 1%, glycine such as 0.2%, 0.3%, 0.4% or 0.5% and the like. Normally, it is aimed that the composition has an osmotic pressure corresponding to a 0.9% w/w sodium chloride solution in water. Moreover, as known by a person skilled in the art, dependent on the specific administration route, pH may be adjusted within suitable ranges centred around pH 7.4. The compositions may be sterilised by conventional, well-known sterilisation techniques. The resulting aqueous solutions may be packaged for use or filtered under aseptic conditions and lyophilised, the lyophilised preparation being combined with a sterile aqueous solution prior to administration.

The compositions may contain pharmaceutically acceptable auxiliary substances as required to approximate physiological conditions, such as pH adjusting and buffering agents, stabilizing agents, preservatives, non-ionic surfactants or detergents, antioxidants, tonicity adjusting agents and the like, for example, sodium acetate, sodium lactate, sodium chloride, potassium chloride, calcium chloride, etc.

According to the present invention, the dose of prostacyclin or an analog thereof is depending on the body weight and general condition of the treated individual. Thus, in the preparation of the treatment, adjustments to the concentration of prostacyclin or an analog thereof may have to be performed. Such adjustments may have to be done immediately prior to surgery, and involve a risk of mistakes. Thus according to the present invention, said pharmaceutical composition comprises an amount of prostacyclin or an analog thereof which allows for a relatively safe and facilitated adjustment of the concentration of prostacyclin or an analog thereof.

In one embodiment of the present invention, said pharmaceutical composition comprises a unit-dose of prostacyclin or an analog thereof for use in treatment as defined herein. A unit dose according to the present invention may be a dose of 0.375 μg to 750 μg, such as 0.750 μg to 150 μg, or such as 150 μg to 225 μg, or such as 225 μg to 275 μg, or such as 275 μg to 325 μg, or such as 325 μg to 375 μg, or such as 375 μg to 425 μg, or such as 425 μg to 475 μg, or such as 475 μg to 525 μg, or such as 525 μg to 575 μg, or such as 575 μg to 625 μg, or such as 625 μg to 675 μg, or such as 675 μg to 725 μg, or such as 725 μg to 750 μg. These doses are intended for intra-operative administration.

In one embodiment of the present invention, said pharmaceutical composition is formulated as a unit-dose of prostacyclin or an analog thereof for use in treatment as defined herein. Such an amount may be adjusted according to the expected length of treatment, which is determined by the length of the period of surgery. A typical length of the period of surgery is 15 min to 360 min, for example an interval of 15 to 60 min, such as 15 min to 30 min, or such as 30 min to 45 min, or such as 45 min to 60 min, or for example an interval of 60 min to 120 min, such as 60 min to 75 min, or such as 75 min to 90 min, or such as 90 min to 105 min, or such as 105 min to 120 min, such as such as 120 min to 135 min, or such as 135 min to 150 min, or such as 150 min to 165 min, or such as 165 min to 180, or for example an interval of 180 min to 240 min, such as 180 min to 195 min, or such as 195 min to 210 min, or such as 210 min to 225 min, or such as 225 min to 240, or for example an interval of 240 min to 300 min such as 240 min to 255 min, or such as 255 min to 270 min, or such as 270 min to 300 min, or for example an interval of 300 min to 360 min such as 300 min to 315 min, or such as 315 min to 330 min, or such as 330 min to 345 min, or such as 345 min to 360 min.

The above doses intended for intra-operative administration may be combined with post-operative administration for any length of time following the surgery. It is preferred that the post-operative administration lasts for 72 hours. It follows that in an embodiment of the present invention, the pharmaceutical composition comprises a unit-dose of prostacyclin or an analog thereof for use in treatment both intra-operatively and/or post-operatively as defined herein. A unit dose according to the present invention for post-operative administration may be a dose of about 86.5 mg to 1728 mg, such as from 172.8 mg to 432.0 mg.

According to the present invention, prostacyclin or an analog thereof may be administered in combination with one or more second active ingredients. Thus, in one embodiment of the present invention, said pharmaceutical composition comprises one or more second active ingredients for simultaneous administration with prostacyclin or an analog thereof.

Kit of Parts

The present invention provides a kit of parts for use in treatment as defined herein. Thus according to the present invention, said kit of parts comprises prostacyclin or an analog thereof or pharmaceutical compositions as defined herein for separate, sequential or simultaneous administration.

According to the present invention, the dose of prostacyclin or an analog thereof is depending on the body weight of the treated individual. In one embodiment of the present invention, said kit of parts include a unit-dose packaging of prostacyclin or an analog thereof for use in treatment as defined herein.

In another embodiment, said kit of parts comprises prostacyclin or an analog thereof in a unit dose of 0.375 μg to 750 μg, such as 0.750 μg to 150 μg, or such as 150 μg to 225 μg, or such as 225 μg to 275 μg, or such as 275 μg to 325 μg, or such as 325 μg to 375 μg, or such as 375 μg to 425 μg, or such as 425 μg to 475 μg, or such as 475 μg to 525 μg, or such as 525 μg to 575 μg, or such as 575 μg to 625 μg, or such as 625 μg to 675 μg, or such as 675 μg to 725 μg, or such as 725 μg to 750 μg.

In one embodiment of the present invention, said kit of parts comprises at least one unit package comprising prostacyclin or an analog thereof in an amount which is suitable for a treatment as mentioned herein. Such an amount may be adjusted according to the expected length of treatment, which is determined by the length of the period of surgery. A typical length of the period of surgery is an interval of 15 min to 360 min, for example an interval of 15 to 60 min, such as 15 min to 30 min, or such as 30 min to 45 min, or such as 45 min to 60 min, or for example an interval of 60 min to 120 min, such as 60 min to 75 min, or such as 75 min to 90 min, or such as 90 min to 105 min, or such as 105 min to 120 min, such as such as 120 min to 135 min, or such as 135 min to 150 min, or such as 150 min to 165 min, or such as 165 min to 180, or for example an interval of 180 min to 240 min, such as 180 min to 195 min, or such as 195 min to 210 min, or such as 210 min to 225 min, or such as 225 min to 240, or for example an interval of 240 min to 300 min such as 240 min to 255 min, or such as 255 min to 270 min, or such as 270 min to 300 min, or for example an interval of 300 min to 360 min such as 300 min to 315 min, or such as 315 min to 330 min, or such as 330 min to 345 min, or such as 345 min to 360 min.

In a preferred embodiment of the present invention, the kit of parts comprises an amount of prostacyclin or an analog thereof suitable for a length of treatment in the range of 60 min to 120 min.

According to the present invention, prostacyclin or an analog thereof may be administered in combination with one or more second active ingredients. Thus, in one embodiment of the present invention, said kit of part further comprises one or more second active ingredients for separate, sequential or simultaneous administration.

In one embodiment of the present invention, prostacyclin or an analog thereof may be comprised in said kit of parts as one or more dosage units of pharmaceutical composition, which can be easily dissolved to obtain a suitable dosage and/or volume for treatment.

The kit of parts according to the present invention may further comprise an aqueous medium to dissolve prostacyclin, an analog thereof, or pharmaceutical composition and other means needed for the administration of prostacyclin or an analog thereof or compositions.

A kit of parts according to the present invention may also comprise instructions for use in a treatment as defined herein.

Methods for Treatment

The present invention relates to method of treatment wherein prostacyclin or an analog thereof is administered to an individual during surgery. Thus, a method according to the present invention comprises one or more steps wherein prostacyclin or an analog thereof is administered by separate, sequential or simultaneous administration to an individual during surgery.

According to the present invention, prostacyclin or an analog thereof may be administered in combination with one or more second active ingredients. Thus, in one embodiment of the present invention, said method of treatment comprises one or more steps wherein a second active ingredient is administered by separate, sequential or simultaneous administration to an individual.

### EXAMPLES

### Example I

The example below demonstrates a method for measuring the state of the glycocalyx and the endothelium in individuals undergoing surgery:

The example is a double bind clinical study in patients undergoing Whipple operation (Pancreaticoduodenectomy). Patients included in the study are randomized and administered either Flolan (prostacyclin analog) in doses of 2 ng/kg/min or saline in equal volume perioperatively.

The protection of glycocalyx measured by enzyme linked immunosorbent assay (ELISA):

Blood is sampled immediately upon arrival, in ethylene-diamine-tetraacetic-acid (EDTA), citrate, heparin (plasma) and serum tubes. Blood samples are obtained without or with minimal stasis (<30 s), ice-cooled, and, immediately after clotting, centrifuged at 2000 g for 10 min. Serum samples are frozen within 1 h of sampling and stored at −80° C. until analyzed.

Soluble markers of glycocalyx degradation (syndecan-1 and glypican-1, hyaluranan) and markers of inflammation, tissue and endothelial damage, endothelial activation, natural anticoagulation and fibrinolysis are measured in uniplicate in plasma samples by commercially available immunoassays according to the manufactures recommendations. Plasma levels of syndecan-1 and other markers indicative of glycocalyx damage including glypican-1, hyaluronan are measured. Plasma levels of markers indicating endothelial cell activation and damage including adrenaline, noradrenaline, ICAM-1, E-selectin, Soluble fms-like tyrosine kinase-1 (also called sFlt-1 or sVEGF R1), sVE-cadherin, angiopoietin 1 (Ang-1), angiopoietin 2 (Ang-2), soluble thrombomodulin (sTM), soluble endothelial protein C receptor (sEPCR), protein C (PC), activated protein C (APC), Antithrombin III (AT) (Antithrombin III), tissue factor pathway inhibitor (TFPI), von Willebrand factor (vWF), tissue-type plasminogen activator (tPA), Factor XIII, histon-complexed DNA fragments, high-mobility group protein B1 (HMGB1), d-dimer, IL-6 and sC5B9 are also measured in order to monitor the level of complications resulting from surgery.

Other Measured Clinical Factors:

The patients are followed pre-surgery, intraoperatively and post-surgery. Data is collected regarding the need for fluid administration perioperatively and/or post-surgery, or the volume of fluid administered perioperatively and/or post-surgery, the weight gain at 12 hours, 24 hours and 48 hours post-surgery, incidence of abdominal compartment syndrome, and need for intensive care therapy including need for pressor support (using drugs such as noradrenaline and dobutamine), ventilatory support, dialysis and treatments of septic complications.

Statistics

Statistical analysis is performed using conventional methods. Patients treated with prostacyclin (Flolan) during surgery are compared to patients who are not treated with prostacyclin (Flolan) during surgery. A reduced level of one or more markers in plasma samples from patients treated with Flolan compared to patients treated with saline shows that the treatment is useful for prevention of capillary leakage which is mediated by a protection of the endothelium and its glycocalyx.

A reduction in one or more of the following a) the total need for fluid administration during surgery and the need for fluid administration post-surgery, b) the weight gain 12 h, 24 h and 48 h post-surgery, c) incidence of abdominal compartment syndrome, d) need for intensive care therapy including need for pressor support (using drugs such as noradrenaline and dobutamine), ventilatory support, dialysis and treatments of septic complications, in patients treated with prostacyclin compared to patients receiving saline shows that the treatment is useful for prevention of capillary leakage.

### Example II

### Demonstration of the Minimal Effects of Low Doses of a Prostacyclin Analog on the Platelet Aggregation and Blood Coagulation in Healthy Volunteers

Six healthy volunteers were administered Flolan (prostacyclin analog) intravenously at a dose of 4 ng/kg/min for 2 h. Blood samples for whole blood viscoelastical assay (Thrombelastography [TEG]) and whole blood platelet aggregation (Multiplate) was obtained before infusion of Flolan, after 60 min infusion of Flolan and after 120 min infusion of Flolan.

With regard to the TEG assay this was performed as recommended by the manufacturer and 340 μl are mixed with 20 μl CaCl 0.2 M (final concentration 11.1 mM in the cup) and kaolin at 37° C. after which the haemostatic activity was recorded as shown in WO 2010/075861.

Whole blood impedance aggregometry was analyzed by the Multiple Platelet function Analyzer (MultiPlate® analyzer). Analysis employing various platelet agonists: ASPI-test (activation by arachidonic acid), COL-test (activation by collagen through the collagen receptor), TRAP-test (activation by TRAP-6 (thrombin-receptor activating peptide) stimulates the thrombin receptor on the platelet surface, and ADP-test (activation by adenosine 5′-diphosphate (ADP) stimulates platelet activation by the ADP receptors). 300 μL adjusted sample was mixed with 300 μL of NaCl (ASPI-test) or NaCl—CaCl₂ (COL-test, TRAP-test, ADP-test) and 20 μL of PLT agonists:

1) TRAP-test: TRAP-6 final concentration, 32 micromol/L,

2) ADP-test: ADP concentration 6.5 micromol/L,

3) COL-test: collagen concentration 3.2 microg/mL,

4) ASPI-test: arachidonic acid concentration (AA), 0.5 mmol/L

MultiPlate continuously records platelet aggregation. The increase of impedance by the attachment of platelets onto the Multiplate sensors was transformed to arbitrary aggregation units (AU) and plotted against time as shown in WO 2010/075861.

Results:

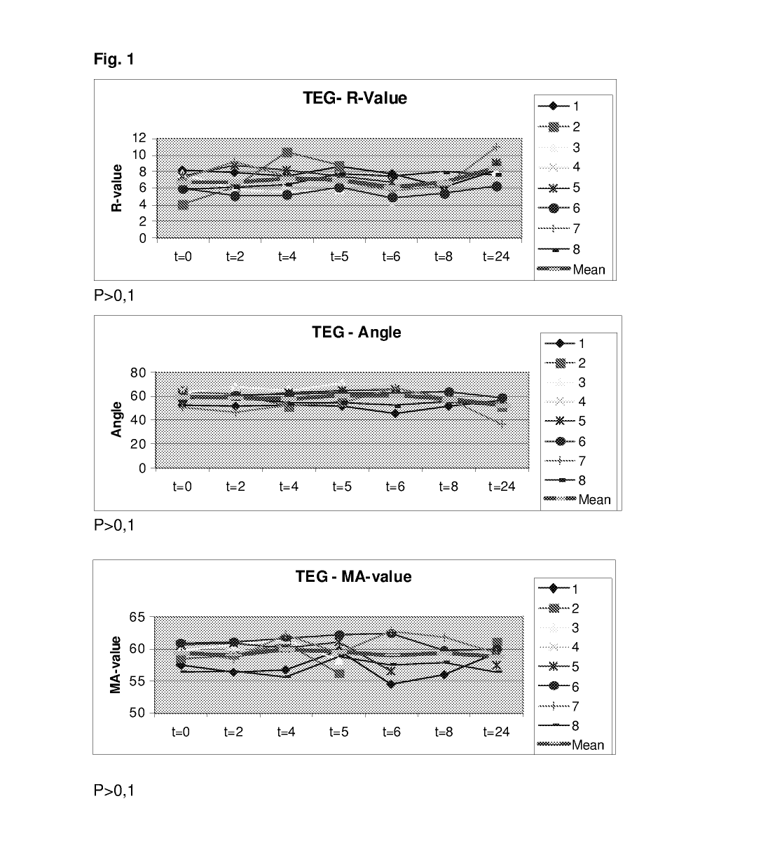

No significant difference was observed when comparing baseline TEG values with samples obtained after 60- and 120 min of Flolan infusion for any of the parameters investigated (R, Angle, MA) in any of the 6 volunteers studied, see FIG. 1.

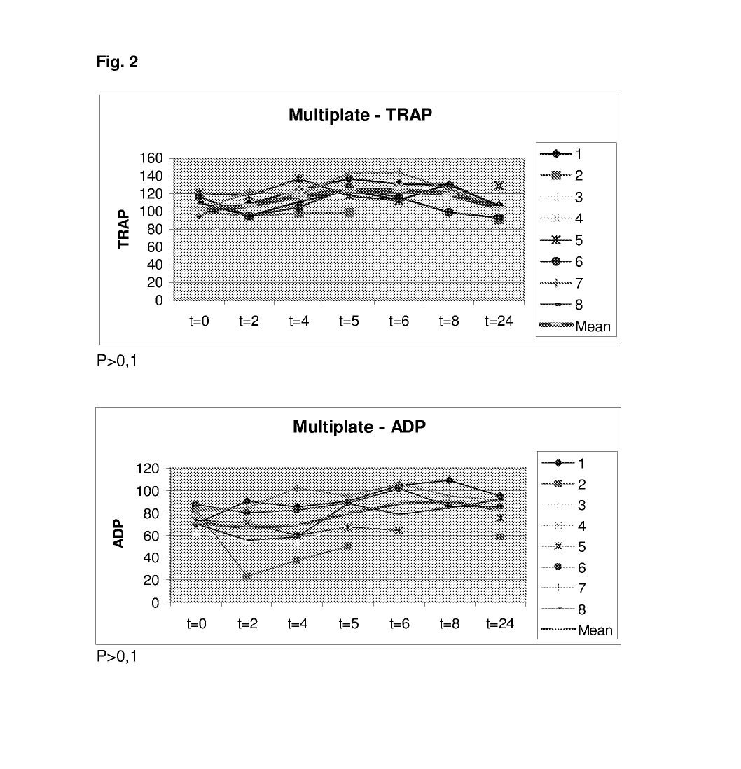

Similarly, no significant difference was observed when comparing baseline Multiplate values with samples obtained after 60- and 120 min of flolan infusion for any of the agonists investigated. FIG. 2 shows values for ADP and TRAP-tests of the 6 volunteers studied.

### Example III

### Endothelial Protective and Anticoagulation Effects of Flolan® Infusion in Healthy Subjects

Study Protocol

Eight healthy volunteers were administered Flolane (Prostacyclin) intravenously at a dose of 4 ng/kg/min for 2 h. Blood samples were analyzed for plasma biomarkers indicative of endothelial cell (thrombomodulin, PAI-1) and glycocalyx (syndecan-1) activation and/or damage, cellular necrosis (histone-complexed DNA fragments, HMGB1) and anticoagulation (protein C, antithrombin, TFPI) at the following time points: Before the infusion (0 h), immediately after ceasing the infusion (2 h) and then 4 h, 5 h, 6 h, 8 h and 24 h after starting the infusion.

The concentration of the individual biomarkers in plasma was analyzed by commercially available ELISA kits according to the manufactures recommendations. Paired t-tests with p-values <0.05 were considered significant.

Results

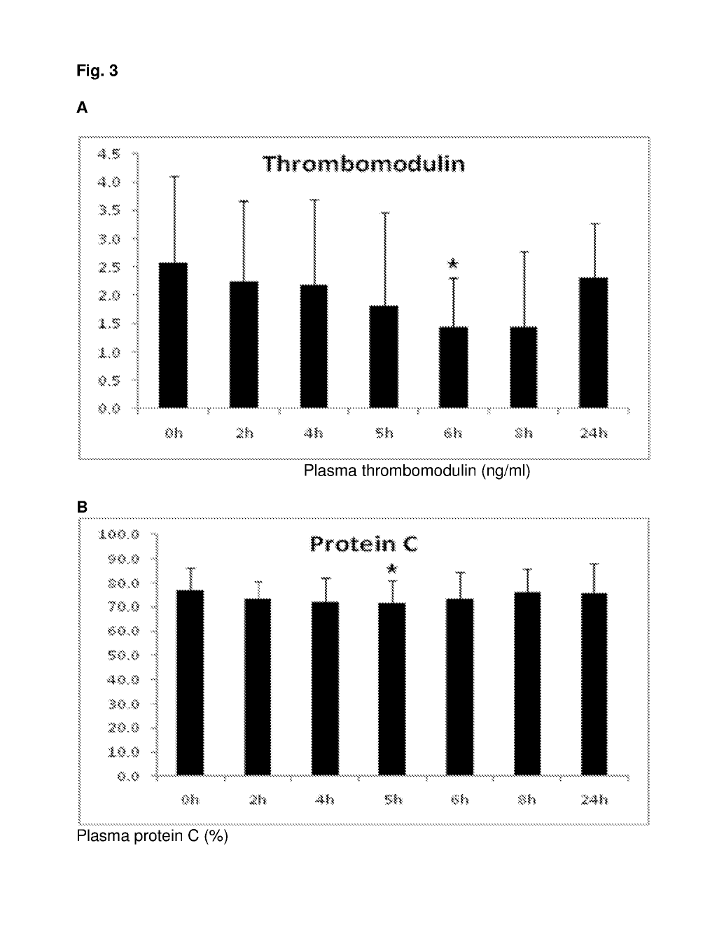

Prostacyclin in the administered dose had an endothelial protective effect evidenced by a marked decrease in the circulating level of thrombomodulin, an effect that seemed to be prolonged and continuing for several hours after ceasing the infusion (FIG. 3A). Furthermore, the circulating level of Protein C decreased in the hours after ceasing the Flolan infusion, indicating that prostacyclin enhanced activation of Protein C (resulting in a decline in the non-activated form of protein C) (FIG. 3B).

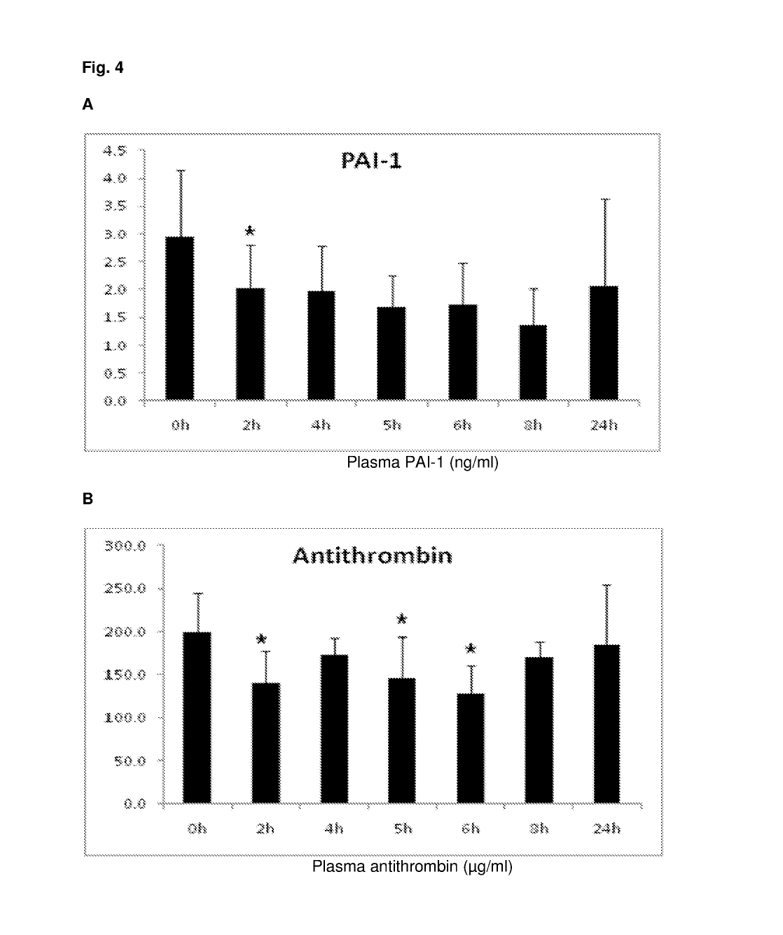

Furthermore, the circulating level of PAI-1, an inhibitor of fibrinolysis shed from the activated endothelium, also declined (FIG. 4A), further indicating that the prostacyclin infusion deactivated the endothelium and enhanced endogenous fibrinolysis. Finally, the circulating level of antithrombin also decreased (FIG. 4B) indicating that a higher amount of this was attached to the endothelial glycocalyx rather than being on a soluble form (FIG. 4B).

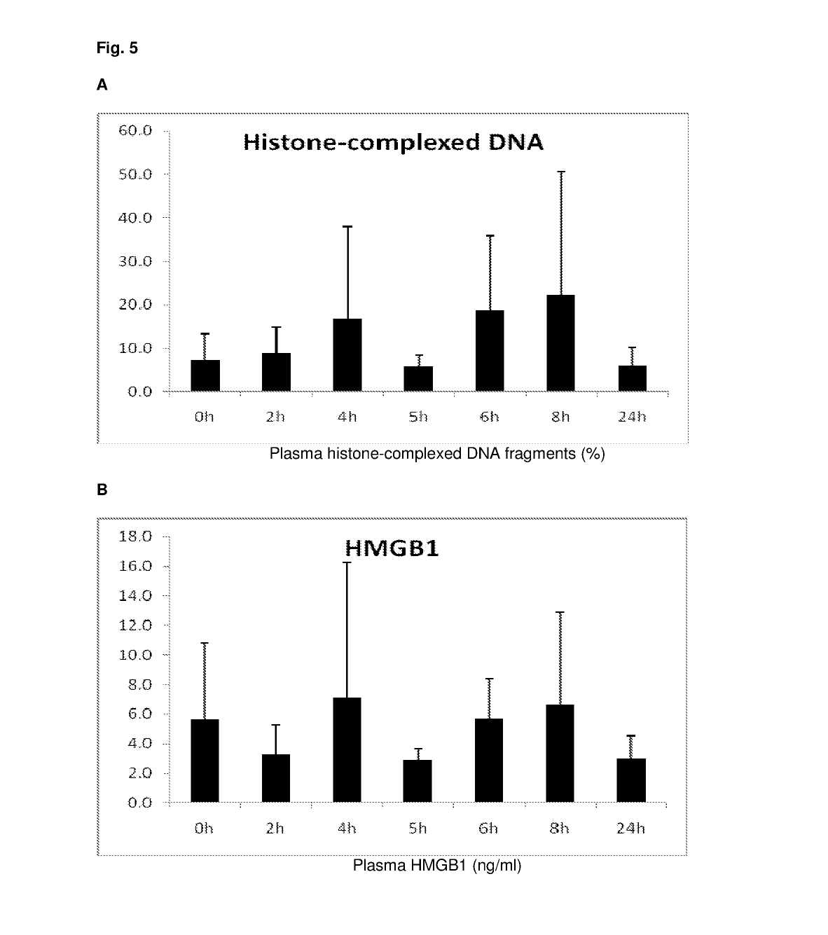

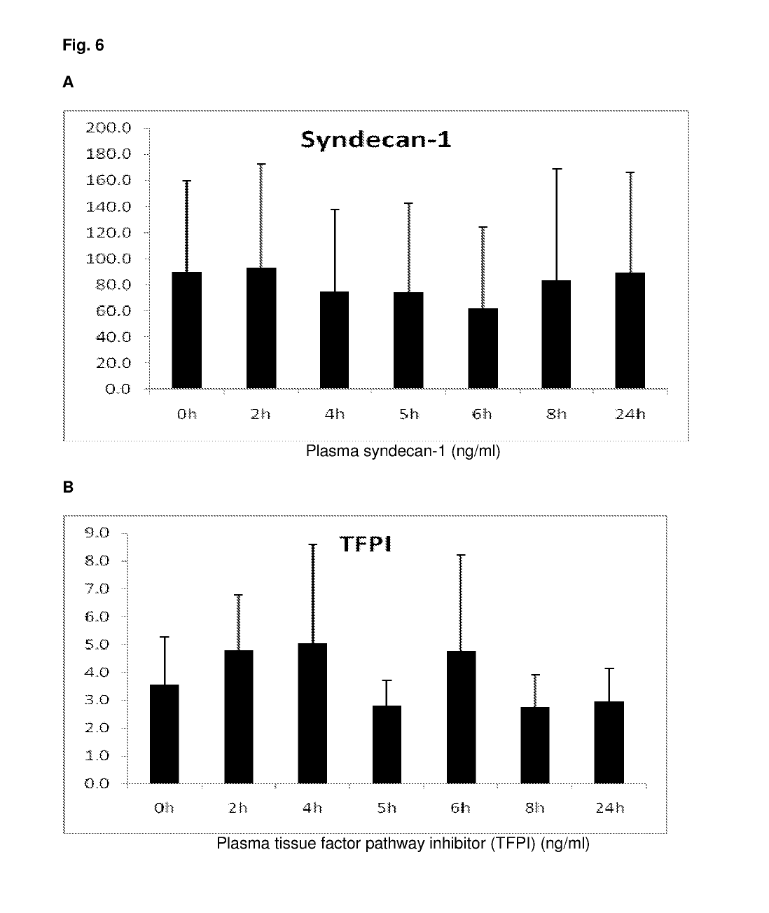

Prostacyclin did not influence the cell necrosis biomarkers (histone-complexed DNA fragments, HMGB1) significantly, though there was a tendency towards a reduction in HMGB1 at 5 h (p=0.056) (FIG. 5A-B). Prostacyclin did not induce or influence glycocalyx shedding (syndecan-1) (FIG. 6A) or affected the circulating level of TFPI (FIG. 6B).

Conclusion

The finding that the administered dose of prostacyclin was associated with concurrent decreases in thrombomodulin and Protein C in healthy individuals is a proof-of-concept of the endothelial protective effect of prostacyclin. Mechanistically, the finding indicates that prostacyclin reduces endothelial release/shedding of thrombomodulin, a recognized marker of endothelial damage, and thereby also increases the amount of protein C that can be activated by/at the endothelium. Activated Protein C exerts a cytoprotective effect on the endothelium through the PAR receptors and high levels of thrombomodulin indicate crude endothelial cell damage and predict high mortality in trauma patients. Given this, this finding identify for the first time an important mechanism by which prostacyclin may improve outcome in patients undergoing major surgery with a high risk of development of capillary leakage syndrome secondary to endothelial modulation.

The finding that PAI-1 decreased along with antithrombin during prostacyclin infusion further indicates that prostacyclin both support fibrinolysis and exerts endothelial protection by increasing antithrombin adhesion to the endothelial glycocalyx. Finally, the finding that prostacyclin did not significantly influence the cell damage biomarkers histone-complexed DNA fragments and HMGB1 (though HMGB1 was reduced non-significantly) and the glycocalyx degradation biomarker (syndecan-1) is expected since the healthy subjects included in this study did not suffer from tissue damage.

### Example IV

### The Effect of Prostacyclin on the Haemostasis, Measured by Thromboelastography and Endothelial Markers by Large Abdominal Surgery

Introduction

In connection with surgical stress, the capillary permeability is increased, resulting in a reduction of the plasma colloid osmotic pressure and loss of intravascular fluid into the interstitial tissue. The patient may subsequently develop hypovolemia and hypotension which is treated by infusion of crystalloids and colloids; this may lead to an increased formation of interstitial edema. A normally functioning vascular endothelium has various proteoglycans and glycoproteins membrane-bound on the luminal side. This layer is designated the glycocalyx. Together with the endothelial cells, plasma proteins and liquid, its primary function is to function as a competent barrier by maintaining and controlling the vascular permeability. Furthermore, the glycocalyx prevents leukocytes and thrombocytes from adhering to the endothelium, thus reducing the development of inflammation and tissue edema. At the whole-body level, the glycocalyx has bound about 700-1,000 ml of plasma (in a 70 kg healthy adult, corresponding to 30% of the total plasma amount) to the endothelial surface, and this non-circulating part of the plasma volume is in dynamic equilibrium with the circulating plasma volume. When the equilibrium between the circulating and non-circulating plasma is disturbed, and the endothelium—in connection with the glycocalyx—is being influenced by stress, the capillary permeability is increased which may result in a capillary leak syndrome (CLS) with subsequent formation of severe interstitial edema.

Prostacyclin (PGI2) is formed and released by normally functioning endothelial cells, and PGI2 protects the endothelial/vascular integrity and is furthermore cytoprotective. Today PGI2 is used for the treatment of critically ill patients with pulmonary hypertension, lung injuries (ARDS) and critical ischemia of the extremities, as well as anticoagulant in filters for dialysis.

Purpose

The purpose of the present study is to clarify the pharmacological effects of continuous infusion of prostacyclin in patients being operated for cancer in the pancreas or in the liver. The purpose of a prospective, randomized study is to investigate
1) the effect on endothelial function (primary endpoint)
2) the effect on the haemostasis
3) the effect on transfusion requirements

Hypotheses

The hypotheses of the project are that:
1) Patients randomized for prostacyclin treatment will have less pronounced endothelial dysfunction/injury measured by endothelial markers, compared to a placebo group.
2) In comparison with placebo, the effect of prostacyclin will not cause more pronounced coagulopathy measured by whole blood analysis (thromboelastography—TEG).
3) The effect of prostacyclin will not affect the need for blood transfusion compared to a placebo group.

Design of the Study

A prospective, randomized, placebo-controlled comparative single-centre study with two treatment groups:
A. Iloprost (Ilomedin®)—administered as an intravenous infusion from the start of the operation until 6 hours after completed operation.
B. Placebo (0.9% saline)—administered as an intravenous infusion from the start of the operation until 6 hours after completed operation.

Material

The patient basis is constituted by patients with cancer in the pancreas or in the liver who have been referred to the KIR-GAS-TRANSPLANT Clinic at Rigshospitalet (Copenhagen University Hospital) for operation. Within one year, it has been planned to include 2×20 patients. Competent adults may be included.

Criteria for Inclusion
1. Men and women above 18 years
2. Undergo Whipple surgery or liver resection
3. Willing to participate in the study after having given informed consent

Criteria for Exclusion
1. Allergy to the test medicine
2. Undergoing treatment with ADP receptor inhibitors, heparin (not as thrombosis prophylaxis), Factor Xa inhibitors, thrombin inhibitors, vitamin K antagonist
3. Autoimmune disorder
4. Intracranial bleeding within the last 6 months
5. Acute coronary disease, myocardial infarction within the last 6 months
6. Acute or chronic congestive heart failure
7. Liver cirrhosis (liver failure)
8. Renal disability requiring dialysis
9. The patient is pregnant or breast-feeding. (In order to exclude pregnancy, women must be postmenopausal (at least 12 months since the last menstruation) or sterilized or having a negative pregnancy test for women of child-bearing age)
10. Participation in another clinical study within the last 30 days.

Effect Parameters

Primary Effect Objectives
Change in the concentration of circulating soluble thrombomodulin (sTM) before operation to 6 hours postoperatively
Change in the concentration of circulating soluble E-selectin before operation to 6 hours postoperatively
Change in the concentration of Syndecan-1 as a measure of glycocalyx degradation from before operation to 6 hours postoperatively

Secondary Effect Objectives
Degree of coagulopathy, evaluated by TEG at the completion of the surgery
Number of blood transfusions from the start of the operation to 6 hours post-operatively

Test Medicine

Iloprost is dispensed as a concentrate to infusion fluid, solution. 1 ml contains 20 μg of Iloprost (as the trometamol salt) in sterile water with a pH of about 8.3. Iloprost is administered as an intravenous dose of 0.5-2 nanogram/kg body weight/minute. In this study, Iloprost 1.0 nanogram/kg/minute is administered from the start of the surgery until discharge from the postoperative ward, but 24 hours at the most. The dose is dissolved in 500 ml of 0.9% saline in accordance with the drug information leaflet. As placebo 500 ml of 0.9% sodium chloride is used, administered in a volume corresponding to that of Iloprost administered.

Randomization

1:1 randomization is used by opening consecutively numbered, opaque envelopes in which the patients are allocated to prostacyclin and placebo, respectively, and this is done at the day of the operation when a person not involved in the treatment of the patient prepares the test medicine preoperatively. The preparation of the test medicine is carried out by skilled staff.

Intervention

In each series the following is given
1. Placebo in the form of a continuous infusion of the same volume as in 2 from the start of the surgery until 6 hours postoperatively. The administration is given in the central vein (CVK).
2. Continuous infusion of prostacyclin 1.0 ng/kg/minute from the start of the surgery until 6 hours postoperatively. The administration is given in the central vein (CVK).

The test is completed for the individual patient 6 hours postoperatively. The test medicine is administered as a continuous infusion by means of an electronic pump system. The preparation and connection of the test medicine must be carried out by staff trained for this. The test medicine is mixed immediately before the operation and must be kept at room temperature, and this mixture has a shelf life of 24 hours.

Blinding

No blinding is made in this study.

Measurements

Study of the Effect on the Endothelium:

The degree of endothelial dysfuncion/activation and injury is studied, measured by a change in biomarkers in plasma from baseline until 6 hours postoperatively. Blood samples are taken from A-cannula preinterventionally (before Iloprost infusion) and then at the completion of the surgery as well as 6 hours postoperatively with subsequent analysis of sTM, soluble E-selectin and Syndecan-1.

Effect on the Haemostasis:

TEG is a whole blood haemostatis analysis reflecting the ability of the blood to coagulate. The analysis identifies various causes of coagulopathy such as lack of coagulation factors or thrombocytes as well as increased fibrinolysis. It has been shown that TEG better reflects the haemostasis and identifies patients with an increased transfusion need compared to conventional coagulation analyses (Johansson et al. 2009). With TEG the haemostasis is investigated before start of the surgery, at the completion of the surgery and 6 hours postoperatively. Furthermore, the number and type (SAG M, FFP, TK) of blood transfusions intra- and 6 hours postoperatively, and also the type (crystalloid/colloid) and amount of infusion fluid intra is recorded—and 6 hours postoperatively.

The following blood samples are taken:
1) Before start of surgery (before Iloprost/placebo infusion is started):
   a. 9 ml of EDTA plasma
   b. 4 ml of citrate plasma
   c. 4 ml of heparin plasma
   d. 4 ml of serum
2) At the completion of the surgery:
   a. 9 ml of EDTA plasma
   b. 4 ml of citrate plasma
   c. 4 ml of heparin plasma
   d. 4 ml of serum
3) 6 hours postoperatively:
   a. 9 ml of EDTA plasma
   b. 4 ml of citrate plasma
   c. 4 ml of heparin plasma
   d. 4 ml of serum

The above analyses (ELISA, TEG) are all established, validated and available in the research laboratory, Section for Transfusion Medicine, Blood Bank 2034, Rigshospitalet.

Statistical Considerations

The primary endpoint of the project is endothelial dysfunction/injury measured by soluble thrombomodulin (sTM). A significance level (alpha) of 5% is used, and the power (1-beta) is set at 80%. When calculating N with the above parameters, least relevant difference (35% mean difference in sTM levels from baseline to discharge from POTA [estimate based on Johansson et al. Ann Surg 2001 and J Trauma 2011 and unpublished data)] and estimated standard deviation of 35% of mean value [estimate based on Johansson et al. Ann Surg 2001 and J Trauma 2011], an attendance of 17 in each arm appears in order to obtain a power of 80%. It was decided to include 2×20 patients.

Side Effects, Risks and Drawbacks

Prostacyclin is a well-known substance which has been used for many years for the treatment of pulmonary hypertension in doses above 10 times the dosage of the present study. In these doses, side effects may be seen as described in the drug information leaflet. By prostacyclin infusion in doses below 2 ng/kg/minute, a few self-limiting hypotensive episodes lasting for 1-2 hours have been described. No other side effects have been described, in particular no bleeding tendency.

All side effects are recorded in the patient's unique CRF, and all incidents will be evident from the final report to the Danish Medicines Agency.

### REFERENCES

Bihari et al., Intensive Care Med. 1988; 15(1):2-7
Di Benedetto et al., Minerva Anestesiol. 2003 June; 69(6):501-9, 509-15.
Dunser: J Int Care Med 2009; 24:293-316
Ghitescu L, Robert M (2002). Microsc Res Tech 57:381-389
Jestice H K et al. Eur J Cardiothorac Surg. 1990; 4(1):40-4.
Kang et al., Anesth Analg. 1985 September; 64(9):888-96.
Van den Berg B M, Vink H, Spaan J A (2003). Circ Res 92:592-594
Reitsma S, Slaaf D W, Vink H, van Zandvoort MAMJ, oude Egbrink, MGA, Eur J Physiol (2007) 454:245-359.
Salooja et al., Blood Coagul Fibrinolysis. 2001 July; 12(5):327-37. Review. Erratum in: Blood Coagul Fibrinolysis 2002 January; 13(1):75.
Schereen et al., Intensive Care Med (1997) 23: 146-158
Zardi et al., International Immunopharmacology 5 (2005) 437-459
Zardi et al., Prostaglandins & other Lipid Mediators 83 (2007) 1-24

1. A compound which is prostacyclin or an analog thereof, for prevention of capillary leakage by intraoperative and/or post-operative administration.

2. The compounds according to claim 1, wherein the prostacyclin or an analog thereof is administered intra-operatively.

3. The compound according to claims 1-2, wherein the prevention of capillary leakage is mediated by protection of the endothelial cells and/or the glycocalyx.

4. The compound according to the previous claims wherein the prevention of capillary leakage is mediated by protection of the glycocalyx.

5. The compound according to the previous claims, for intraoperative and/or post-operative administration in a surgery selected from a gastroenterologial, thoracic, orthopaedic, urological, gynaecological, plastic, cosmetic or reconstructive procedure.

6. The compound according to the previous claims, for intraoperative and/or post-operative administration in surgery not related to ischemia, cardiovascular diseases, trauma, transplantation or insertion of stents and grafts.

7. The compound according to the previous claims, wherein said compound is selected from the group of PGI2, PGX, prostacyclin (Epoprostenol) or variants thereof, beraprost sodium, epoprostenol sodium, iloprost, iloprost in combination with bosentan, iloprost in combination with sildenafil citrate, treprostinil, pegylated treprostinil, treprostinil diethanolamine, treprostinil sodium, 2-{4-[(5,6-diphenylpyrazin-2-yl)(isopropyl)amino]butoxy}-N-(methylsulfonyl)acetamide, {4-[(5,6-diphenylpyrazin-2-yl)(isopropyl)amino]butoxy}acetic acid, 8-[1,4,5-triphenyl-1H-imidazol-2-yl-oxy]octanoic acid, isocarbacyclin, cicaprost, [4-[2-(1,1-Diphenylethylsulfanyl)-ethyl]-3,4-dihydro-2H-benzo[1,4]oxazin-8-yloxy]-acetic acid N-Methyl-d-glucamine, 7,8-dihydro-5-(2-(1-phenyl-1-pyrid-3-yl-methiminoxy)-ethyl)-a-naphthyloxyacetic acid, (5-(2-diphenylmethyl aminocarboxy)-ethyl)-a-naphthyloxyaceticacid, 2-[3-[2-(4,5-diphenyl-2-oxazolyl)ethyl]phenoxy]acetic acid, [3-[4-(4,5-diphenyl-2-oxazolyl)-5-oxazolyl]phenoxy]acetic acid, bosentan, 17[alpha],20-dimethyl-[DELTA]6,6a-6a-carba PGI1, and 15-deoxy-16[alpha]-hydroxy-16[beta],20-dimethyl-[DELTA]6,6a-6a-carba PGI or derivatives thereof.

8. The compound according to the previous claims, wherein said compound is epoprostenol or iloprost or derivatives thereof.

9. The compound according to the previous claims, for administration in doses of 0.5 ng/kg/min to 4.0 ng/kg/min.

10. The compound according to the previous claims for parental administration.

11. The compound according to the previous claims for continuous intravenous administration.

12. The compound according to the previous claims for single bolus administration or repeated dose administration, or subcutaneous administration.

13. The compound according to the previous claims wherein said intra-operative treatment period has a length of 15 to 360 min.

14. The compound according to claim 13 wherein said treatment period has a length of 60 to 120 min.

15. The compound according to the previous claims wherein the treatment period includes the intraoperative treatment period and a post-operative period of up to 72 hours.

16. The compound according to the previous claims, wherein said treatment decreases or reduces risk of increased levels in a bodily sample of one or more markers selected from syndecan-1, glypican-1 and hyaluronan.

17. The compound according to the previous claims, wherein said treatment decreases or reduces risk of increased levels in a bodily sample of one or more markers selected from adrenaline, noraderenaline, ICAM-1, E-selectin, Soluble fms-like tyrosine kinase-1 (sFlt-1), sVE-cadherin, angiopoietin 1 (Ang1), angiopoietin 2 (Ang-2), soluble thrombomodulin (sTM), soluble endothelial protein C receptor (sEPCR), protein C (PC), activated protein C (APC), Antithrombin III (AT), tissue factor pathway inhibitor (TFPI), von Willebrand factor (vWF), tissue-type plasminogen activator (tPA), Factor XIII, histon-complexed DNA fragments, high-mobility group protein B1 (HMGB1)) d-dimer, IL-6 and sC5B9.

18. The compound according to the previous claims, wherein said treatment decreases or reduces risk of one or more of the following factors:
a) need for fluid administration perioperatively and/or post-surgery, or the volume of fluid administered perioperatively and/or post-surgery,
b) weight gain 12 hours, 24 hours, 48 hours and 72 hours post-surgery compared to pre-surgery,
c) incidence of abdominal compartment syndrome pre-surgery,
d) need for supportive therapy such as intensive care therapy, pressor support, ventilatory support, dialysis and treatments of septic complications,
e) occurrence of single/multiple organ failures selected from the group of organ failures in the central nervous system, lungs, heart, gastrointestinal system, kidneys, liver and haematological systems, and
f) coagulopathy.

19. The compound according to the previous claims, for treatment wherein the thrombelastography values measured during or after treatment in a citrated blood sample activated by kaolin are within the ranges a) R between 3.0 to 8.0 minutes, b) Angle between 55° and 78°, and c) MA between 51 mm to 69 mm.

20. The compound according to the previous claims, wherein the treatment has discrete or minimal vasodilation effects on the microcirculation.

21. The compound according to the previous claims, for treatment wherein the aggregation units measured by multiplate electrical impedance aggregometry during or after treatment are in the range of 40 to 200.

22. A pharmaceutical composition for treatment or prevention of capillary leakage, which comprises a compound as defined by claims 1 to 21.

23. The pharmaceutical composition according to claim 22 wherein the compound mediates a protection of the endothelial cells and the glycocalyx.

24. The pharmaceutical composition according to claim 22 wherein the compound mediates a protection of the endothelial glycocalyx.

25. The pharmaceutical composition according to claims 22 to 24 wherein said compound is comprised in a dose of 0.375 μg to 750 μg.

26. The pharmaceutical composition according to claims 22 and 25 wherein said compound is comprised in a dose suitable for a period of treatment which is 15 min to 360 min.

27. The pharmaceutical composition according to claims 22 to 26 further comprising one or more second active ingredients.

28. The pharmaceutical composition according to claims 22 to 27, wherein at least one second active ingredient is an agonist or antagonist of adrenergic receptors.

29. The pharmaceutical composition according to claims 22 to 28, wherein at least one second active ingredient is Antithrombin III (AT), hydrocortisone, glucocorticoids, N-acetylcysteine, plasma, valproate or albumin.

30. A kit of parts for treatment or prevention of capillary leakage comprising a compound as defined in claims 1 to 21 or pharmaceutical composition as defined in claims 22 to 29.

31. A method for treatment or prevention of capillary leakage comprising a step wherein a compound as defined in claims 1 to 22 or a pharmaceutical composition as defined in claims 22 to 29 is administered intraoperatively and/or post-operatively by separate, sequential or simultaneous administration to an individual.

32. The method for treatment according to claim 31, further comprising a step wherein one or more second active ingredients is administered separately, sequentially or simultaneously to an individual.
